(19) United States
(12) Patent Application Publication
(10) Pub. No.: US 2006/0206028 A1
(43) Pub. Date: Sep. 14, 2006

(54) Apparatus and method for ablating deposits from blood vessel

(75) Inventors: Yee-Chun Lee, Mountain View, CA (US); Qi Yu, City of Industry, CA (US)

Correspondence Address: CHEIN-HWA S. TSAO, 6684 MT PAKRON DRIVE, SAN JOSE, CA, 95120, US

(73) Assignee: Qi Yu, City of Industry, CA (US)

(21) Appl. No.: 11/077,942

(22) Filed: Mar. 11, 2005

(51) Int. Cl. A61B 8/14

(52) U.S. Cl. 600/471

(57) ABSTRACT

An apparatus for ablating deposits along the blood vessel of human and animals is disclosed. The apparatus has an extracting and pressurizing unit for extracting blood from a supply vessel and pressurizing it plus a downstream delivering and injecting unit for delivering and injecting the filtered and pressurized source blood into a blood vessel under treatment. Besides inducing a blood circulation and having ablation devices like ultrasound and RF heating, the apparatus ablates the deposits from a nearby portion of the vessel. The characteristics of selective ablation and self-termination make the proposed apparatus safe and effective in treating early-stage atherosclerosis. A DC discharging device can be included to neutralize excess surface charge generation on the wounded healthy tissues following ablation for disinfection and anti-inflammation. Placement of the blood extracting point just downstream of the blood injecting point insures thorough collection and removal of blood-clogging plaque and calcification fragments.

### CROSS REFERENCE TO RELATED APPLICATION

### BACKGROUND OF THE INVENTION

1. Field of the Invention

The present invention relates generally to the field of medical apparatus. More particularly, this invention relates to a new apparatus for clearing and removing undesirable deposits from an inner blood vessel wall.

2. Description of the Related Art

Atherosclerosis, or the “hardening of the artery”, is generally associated with the drastic shrinkage of the inner diameter of the artery through prolonged deposition or degenerative accumulation of fatty substances, such as cholesterol, etc., on the inner layer of the artery wall. In real life, the biological process accompanying atherosclerosis is a lot more complex, including a self-healing mechanism of the human or animal body that attempts to minimize the constriction of the artery, called stenosis in medical terminology. Here, the self-healing mechanism functions by externally enlarging the artery, or “remodeling” in medical terms. The constituents of these prolonged depositions, called atheroma, include microphage cells, cellular debris of dead cells and living cells, as well as the fibrous tissue covering of the atheroma itself Over time, calcification can also occur between the atheroma layer and the underlying smooth muscle cell layer of the vessel wall.

The combination of the calcification layer, atheroma and the fibrous tissue cap, jointly called “atheromatous plague”, will grow with time and eventually causes the inner diameter to narrow when the external enlargement of the artery wall can no longer keep up with the growth. But even before this happens, the very existence of the atheroma can cause the artery wall to stiffen and becomes fragile due to the aforementioned calcification. Structurally, atheroma is a foamy substance as a result of the vesicular buildup and its final physical property is soft, fragile while inelastic.

At an advanced stage, the fibrous cap of the atheroma layer is prone to rupture. The cause can be nothing more than a slightly stronger than normal heart beat. Upon rupture, the fragmented tissues can collect platelets, which are cell-like structures resembling glues in that, whenever they come in contact with collagen (a kind of strong, white connective tissue found in skin, etc.), they will activate the blood clotting mechanism to “thicken the blood” and to form “fibrin clots” which protrude into the interior of the artery vessel and cause a temporary stenosis. In addition, the fragmented calcification deposits and tissue debris, if their diameters happen to be greater than 5 micron (1 micron=10⁻⁶ meter), can clog the micro veins leading to debility and sometimes sudden death. Therefore, any measure that attempts to remove these atheromatous plaques should insure that it will be able to either immediately remove the plaque fragments from the blood stream or to pulverize the plaque tissue and calcification debris to particle sizes much smaller than 5 micron. Additionally, provisions should be made to counter the tendency of blood clot formation as a result of the pulverization of the atheromatous plague.

Numerous medical equipments and techniques are available today for unblocking coronary arteries blocked by the atheromatous plaque, a build-up of cholesterol and other fatty substances on the inner lining of an artery. Chief among them are balloon angioplasty, laser angioplasty, stents, rotational atherectomy, directional atherectomy and transluminal extraction atherectomy. Except for balloon angioplasty, typically performed only after enough plaque has been removed by other techniques, the other equipments and techniques are highly invasive in nature and are used in situations where major coronary arteries are blocked hence require speedy reopening. For the removal of early stage plaques, it is generally too risky to use such highly invasive techniques.

During an actual medical procedure, each of these techniques typically uses a thin flexible tube, called catheter, that is guided to thread its way through a major artery until its tip reaches the diseased area within the artery wall. For guidance, a guide wire is typically inserted first before the catheter. The catheter is then passed over the guide wire to reach the target area.

As a more detailed example of the prior art, laser angioplasty, also called laser atherectomy, utilizes a surgical laser attached to the tip of a catheter that emits short pulses of intense laser light that ablates the atheromatous plaques that block the artery. To avoid a concurrent damaging of the artery walls by the laser beam, the patient is injected with tagged antibodies in advance that theoretically can attach to plaque molecules thus guiding the laser pulses to plaque molecules. However, the risk of laser scarring healthy artery wall tissues is still significant. Examples of the risks associated with laser atherectomy include artery perforation, cardiac arrhythmias, genetic mutation caused by ultra violet (UV) radiation from a UV laser, restenosis, toxic gas leakage from the equipment, laser-induced vapor bubbles that can damage artery walls and vascular spasm. Following the laser procedure, an X-ray contrast dye is injected into the blood stream to determine whether balloon angioplasty is required under X-ray imaging of the intimate areas of treatment. Balloon angioplasty utilizes a catheter with a folded balloon attached to its end. When hydraulically inflated, the balloon compresses the plaque and stretches the artery wall to expand. Simultaneously, a “stent”, an expandable mesh tube enclosing the balloon, expands with the balloon and is then, upon deflation and removal of the balloon, left behind. The stent functions to support the newly stretched open position of the artery from inside.

As a second example of the prior art, rotational atherectomy is often used in lieu of laser angioplasty to remove coronary artery blockage. It utilizes a high speed (around 200,000 rpm) rotational elliptical “burr” coated with microscopic diamond to break up the blockage into fragments, often smaller than red blood cells, which then pass harmlessly into the blood circulation. The diamond coated burr is welded to a flexible drive shaft that tracks along a central guide wire. The drive shaft is housed in a thin sheath that in turn is connected to an advancer that contains a high-pressure, air-powered turbine. Meanwhile, a continuous infusion of saline facilitates dissipation of the heat generated by the spinning drive shaft and minimizes arterial spasm. Burr sizes range from 1.25 to 2.5 mm (millimeter) in diameter. Due to the high rotational speed and the hardness of the diamond bits, the risk of tearing of an artery and bleeding around the heart is significant, despite the claimed theory of “differential cutting” stating that a rotational atherectomy equipment driven by compressed air can preferentially ablate away the atheromatous plaque while leaving the intimate healthy issue intact. Notwithstanding this risk, a spinning diamond coated burr can not harm untouched tissue. In contrast, during laser angioplasty the laser energy does have a longer reach and can vaporize tissue some distance away. Hence laser angioplasty is inherently more dangerous, which explains why it has not been used as frequently as other invasive procedures. Rotational atherectomy is particularly effective in treating heavily calcified and inelastic, or long lesions.

As a third example of the prior art, directional atherectomy is similar to rotational atherectomy. Directional atherectomy uses a special catheter whose tip contains a small cylindrical rotating steel cutting blade encased in a metal housing that has an opening on one side and a balloon on the other side. A tiny plastic cone at the end of the tip collects the shaved-off plaque fragments. The cutting blade rotates at around 2000 rpm to shave off the plaque from the arterial wall. The shaved-off plaque fragments are immediately collected in the plastic cone. Following a later withdrawal of the catheter, the plaque fragments are then cleared from the cone. The risk of injury is lower for directional atherectomy than for both laser angioplasty and rotational atherectomy. Given a proper positioning of the direction of the blade opening, the resulting risk of physical injury is small. However, directional atherectomy is not as effective in removing heavily calcified plaques due to its lower spinning speed and the lower hardness of steel.

Transluminal extraction atherectomy is yet another prior art procedure involving a special catheter tipped with a hollow tube and rotational blades. Transluminal extraction atherectomy differs from directional atherectomy in that the hollow tube allows the plaque fragment debris to be suctioned out of the body through the tube. Otherwise, its benefits and associated risks are similar to that of directional atherectomy.

Typically, highly invasive procedures such as laser angioplasty and rotational atherectomy are only employed upon serious blockage of the coronary artery. For less serious cases wherein the coronary arteries are merely “narrowed” or “hardened”, balloon angioplasty is usually performed instead. This is because the more invasive procedures work best when the plaques protrude into the lumen, the interior space, of the blood vessel. A plaque that leans flat against the arterial wall is much harder to remove with either laser or mechanical cutting without risking serious injury to the blood vessel itself owing to the proximity of the diseased area to the healthy vessel wall muscular tissue.

The drawback of balloon angioplasty is that, by itself, it does not remove plaque. What it does is merely reshaping the vessel wall through stretching and compressing the plaque, followed by “stenting” to maintain the newly formed shape. The trouble with this approach is that the procedure does not stop or even slow down the atherosclerosis (hardening of the arteries) as the plaque itself tends to attract more deposition of fatty substances onto it. Furthermore, the calcification of the interface between the plaque tissue and the inner wall lining (intima) continues unabated and could even accelerate in the presence of the calcified layer already there. This continued calcification tends to make the artery wall inelastic and fragile even if no significant narrowing of the arteries has taken place. Only through removal of the plaque and the calcified layer can atherosclerosis be effectively slowed down or reversed.

Removal of plaque with laser or mechanical cutting brings on additional complications. Specifically, the torn diseased tissue fragments can carry charges and, as such, they can activate the body's blood clotting system intended for preventing blood loss due to external bleeding. Hence, the activation can cause the blood to coagulate, or to become thickened, as well as becoming inflamed. Both blood coagulation and inflammation of the torn inner lining can lead to additional clogging and narrowing of the blood vessel, further compounding the problem.

Yet another potential complication accompanying the high-speed pulverization process is that some of the generated plaque fragments may not be small enough to travel through the blood stream, causing clinically significant emboli that could be deadly. This can be especially serious for a procedure like the rotational atherectomy. Regarding this risk, directional atherectomy and transluminal extraction atherectomy are often safer as they tend to capture more of the plaque fragments before the rest are allowed to travel through the blood stream. However, their inability to effectively pulverize heavily calcified tissue also increases the danger of letting comparatively large calcified fragments travel through the blood stream, possibly causing an instant death.

Recent evidence suggests that during the slow, gradual buildup of atheromatous plaques, small plaque ruptures can sometime occur which in turn cause a sudden increase in plaque burden owing to the accumulation of blood clotting substance. This can take place even at locations of heavy plaque buildup yet with little or no lumen narrowing. Generally a plaque becomes vulnerable to rupture when it starts to grow rapidly and has a thin fibrous cover separating it from the bloodstream inside the lumen. Plaque rupture occurs when the fibrous plaque cover is torn. Upon rupture, tissue fragments get spilled into the bloodstream as debris. The debris is frequently too large to pass through capillaries hence obstructs smaller downstream branches of the blood vessel. Rupture may also allow bleeding from the lumen into the inner tissue of the plaque, making it expand rapidly and protrude into the lumen of the artery resulting in lumen narrowing or even obstruction. Additionally, blood clotting activated by the tearing of the fibrous plaque cover can rapidly block the passage of the artery thereby stopping the blood flow to the tissue the artery supplies.

By now it should become clear that none of the cited prior art medical equipments and techniques can satisfactorily address all of the risks and problems just described. While both laser and rotational atherectomy can be effective albeit risky for the ablation of late stage plaque blockage, they are nearly ineffective in treating early and mid-stage plaque formation. This is particularly troublesome in view of the fact that mid-stage vulnerable plaque formation with minimum lumen intrusion is now clinically considered to be even more dangerous owing to its tendency to rupture spontaneously, leading to immediate and severe heart attack or even instant death.

Balloon angioplasty and stent are, on the other hand, minimally intrusive and can be considered safe for treatment of mid-stage plaque formation. However, as they do not really remove plaque from the inner lining of the artery wall, they only tend to temporarily reduce the symptom of lumen narrowing. Extensive human clinical studies have failed to show clinically significant improvement of the mortality rate of the patients who had undergone the angioplasty and stent operations. For those patients who had mechanical atherectomy performed on them to treat late-stage atherosclerosis, the inability of the high-speed pulverization process used by the atherectomical instruments to cut the plaque tissue into small enough fragments is a cause for real concern considering its risk of emboli.

Equally importantly, none of the cited prior art medical equipments and techniques can address the problem of early-stage plaque formation. The inability of balloon angioplasty and stent to remove plaque renders them essentially ineffective in treating the early-stage plaque. The potential of great harm to the artery wall tends to rule out laser and rotational atherectomy. It is unlikely that the relatively safe directional and transluminal extraction atherectomy procedures would be able to shave off the comparatively shallow plaque layer without shaving into the healthy artery wall tissue, tearing the wall in the process.

In view of the above, it is highly desirable to have a device capable of selective fine-grain pulverization of the atheromatous plaque in a self-terminating manner without causing harm to the healthy blood vessel tissue and capable of performance in a minimally invasive fashion. The device should also be able to mitigate undesirable effects from the charge buildup from torn diseased tissue fragments during the treatment to disinfect and promote healing, and to prevent the natural tendency of the blood to coagulate in the presence of a wound resulting from the ablation of the diseased tissues. Last, but not least, the device should facilitate, during operation, the removal of the plaque residues and the collection and removal of plaque fragments that are too large to safely pass through the blood stream.

### SUMMARY OF THE INVENTION

An apparatus is proposed for the cleaning and removal of undesirable deposits, for example calcified deposits or fatty substances, on the inner lining of a blood vessel wall of human and animals. The resulting benefit can include slowing and reversing the advancement of atherosclerosis and other related diseases. Hence, the proposed apparatus can be used for treatment of various sections of the arterial system such as the internal carotid, the left and right common carotid, the coronary arteries, the superior mesenteric, the external iliac and various peripheral arteries. The apparatus can also treat various sections of the venous system such as the internal jugular, the external jugular, the left brachiocephalic, the inferior vena cava, the common iliac and various peripheral veins. The apparatus includes a blood extraction and pressurization unit for extracting blood from a supply blood vessel, filtering it to rid the extracted blood of undesirable substances, pressurizing the filtered blood for re-injecting it into a receiving blood vessel under treatment hence inducing a concomitant blood circulation as well as propelling the undesirable deposits downstream.

The proposed apparatus further includes a delivery tube, a secondary manifold and an injector nozzle in communicative connection with the blood extraction and pressurization unit for delivering and injecting the pressurized source blood into the blood vessel under treatment.

The blood extraction and pressurization unit further includes a primary manifold, which in turn includes a primary inlet, a primary outlet and a pumping device that interconnects the primary inlet and the primary outlet for receiving and pressurizing the extracted source blood.

The blood extraction and pressurization unit further includes a tertiary manifold that includes a tertiary outlet, at least one suction needle for piercing the supply blood vessel and drawing the source blood from there and a suction tube that interconnects the tertiary outlet and the primary inlet for delivering the extracted source blood to the primary manifold.

The blood delivering and injecting unit further includes at least one secondary manifold, in communicative connection with the delivery tube and the injector nozzle, for buffering and filtering the pressurized source blood before its injection through the injector nozzle.

The primary manifold further includes a primary storage with an inline filter for temporarily storing and filtering the extracted blood from the supply blood vessel. The primary storage in turn includes two chambers interconnected through the pumping device so that one chamber stores lower pressure blood and the other chamber stores higher pressure blood.

The primary manifold may further include an electrical subsystem that in turn includes a Radio Frequency (RF) generator for the generation of drive power for the pumping device, drive signal for an ultrasonic power transducer at one or more frequencies, a Direct Current (DC) power source and a drug container for storing and metering an auxiliary drug such as an anticoagulant drug, etc. into the blood stream.

The secondary manifold further includes an ultrasonic power transducer to convert the incoming RF power from the electrical subsystem into an ultrasonic power emission that propagates within the blood stream for the ablation of the undesirable deposits and diseased tissues inside the blood vessel under treatment through pulverization and emulsification, with further filtration with the aforementioned inline filter, into particulates of fine enough size to safely pass through the blood circulation system. While the underlying physics and application of ultrasound-induced pulverization and emulsification process in, for example, the medical treatment of cataract and the cleaning of semiconductor wafers have been established, no prior art systems known to us perform the ablation of blood vessel deposits through such a pulverization and emulsification process.

The frequency of the incoming RF power is made to periodically vary through a pre-determined range so as to tune the ultrasonic power emission to the various mechanical resonances of the calcified tissue of the undesirable deposits thus further enhancing the ability to shatter and pulverize the calcified tissue.

The frequency components and their respective power levels of the incoming RF power can be selected such that the corresponding ultrasonic power emission exhibits a spatially slowly varying standing wave pattern thereby achieving a more spatially uniform pulverization of the deposited plagues.

The wavelength and power of the ultrasonic power emission can be further adjusted to generate cavitations within the blood that preferentially shatter hardened diseased regions based upon their inelasticity while leaving healthy, elastic blood vessel tissues unaffected.

The wavelength and power of the ultrasonic power emission can be further modulated to match a range of natural resonant frequencies of the hardened diseased regions to realize a more effective ablating process.

The range of the above natural resonant frequencies can be further limited to those of the inelastic diseased region to make the ablating process self-terminating in that, once the inelastic diseased regions are removed and flushed away, the corresponding ultrasound pulverization and emulsification actions automatically terminate.

The secondary manifold further includes an electrode affixed to the injector nozzle and powered by the electrical subsystem for discharging charges to neutralize excess opposite-sign charges generated by the tearing of healthy or diseased tissues during the ablating process.

In addition, the secondary manifold also includes an injector for discharging and mixing an anticoagulant drug into the blood stream. Alternatively, the anticoagulant drug can be premixed into the blood in the primary manifold before its delivery to the secondary manifold to be injected into the bloodstream.

Further, the secondary manifold can include a heating device to provide localized heating to destroy diseased tissues.

Additionally, the secondary manifold can include an injector mechanism for injecting a radio-contrast substance to enable the examination of the blood vessel under treatment using X-rays. This injector mechanism can be collocated with the anticoagulant drug injector or, alternatively, it can be a separate injector mechanism either within the secondary manifold or within the primary manifold.

Additionally, the secondary manifold can further include an ultrasound imaging device located close to the injector nozzle for illuminating and examining an illuminated ultrasound image of the blood vessel interior under treatment.

Additionally, the secondary manifold can further include a foldable balloon that, when inflated by the pumping device, substantially blocks the lumen of the blood vessel under treatment within a safety stretch limit while the inflated foldable balloon gets simultaneously pushed along the blood vessel under treatment and functions to prevent an undesirable back flow of the pressurized source blood.

### BRIEF DESCRIPTION OF THE DRAWINGS

Various other objects, features and attendant advantages of the present invention will become fully appreciated as the same becomes better understood when considered in conjunction with the accompanying drawing, in which like reference characters designate the same or similar parts throughout the several views, and wherein:

FIG. 1 illustrates an embodiment of the proposed apparatus of the present invention as applied to a human or animal circulatory system to clean and remove plaques deposited on the inner lining of a blood vessel;

FIG. 2 illustrates a part of a blood extracting and pressurizing unit called a tertiary manifold used to extract blood from the vein or artery of a human or an animal;

FIG. 3 illustrates an embodiment of a primary manifold that, in addition to providing the functions of drug administration, charge signal and RF signal generation, uses a van pump for blood pressurization and filtering;

FIG. 4 illustrates another embodiment of the primary manifold that is equipped with a gear pump;

FIG. 4A illustrates an improvement of the blood filtration within the primary manifold using power transducers located upstream of an inline filter for emitting ultrasonic power into the blood to pulverize and emulsify undesirable substances of the extracted source blood;

FIG. 4B illustrates an overview of the primary manifold embodied with the above improvement of the blood filtration using power transducers in combination with a van pump;

FIG. 4C illustrates another overview of the primary manifold embodied with the above improvement of the blood filtration using power transducers in combination with a gear pump;

FIG. 5 illustrates a front end of a blood delivering and injecting unit of the proposed apparatus that includes a guide wired injector head with an injector nozzle; FIG. 6 illustrates an embodiment of an ultrasonic power transducer head with its electrical driving signal delivered through a waveguide structure;

FIG. 7 illustrates a front portion of the blood delivering and injecting unit called secondary manifold having a blood pressure isolating balloon in this particular embodiment;

FIG. 8 illustrates the placement of the injector nozzle and the secondary manifold having the blood pressure isolating balloon inside a blood vessel under treatment during an ablating procedure;

FIG. 9A illustrates an ultrasonic cavitation process together with its initial interaction with a plaque along the inner blood vessel wall under treatment during the ablating process;

FIG. 9B illustrates a mid stage interaction between the ultrasonic cavitation and the undesirable deposit during the ablating process;

FIG. 9C illustrates a late stage interaction between the ultrasonic cavitation and the undesirable deposit during the ablating process;

FIG. 10 illustrates the excavation of post-pulverization plaques and calcified debris away from a diseased area of the blood vessel under treatment;

FIG. 11 illustrates the neutralization of surface negative charges atop a newly formed tissue wound by positive charge emission from a charge emitting electrode located inside the injector nozzle; and

FIG. 12A and FIG. 12B together illustrate a further improvement of the present invention using a dual tube concept with an end ultrasonic cavity as the front portion of the blood delivering and injecting unit.

### DETAILED DESCRIPTION OF THE PREFERRED EMBODIMENTS

In the following detailed description of the present invention, numerous specific details are set forth in order to provide a thorough understanding of the present invention. However, it will become obvious to those skilled in the art that the present invention may be practiced without these specific details. In other instances, well-known methods, procedures, materials, components and circuitry have not been described in detail to avoid unnecessary obscuring aspects of the present invention. The detailed description is presented largely in terms of simplified two dimensional views. These descriptions and representations are the means used by those experienced or skilled in the art to concisely and most effectively convey the substance of their work to others skilled in the art.

Reference herein to “one embodiment” or an “embodiment” means that a particular feature, structure, or characteristics described in connection with the embodiment can be included in at least one embodiment of the invention. The appearances of the phrase “in one embodiment” in various places in the specification are not necessarily all referring to the same embodiment, nor are separate or alternative embodiments mutually exclusive of other embodiments. Further, the order of process flow representing one or more embodiments of the invention do not inherently indicate any particular order nor imply any limitations of the invention.

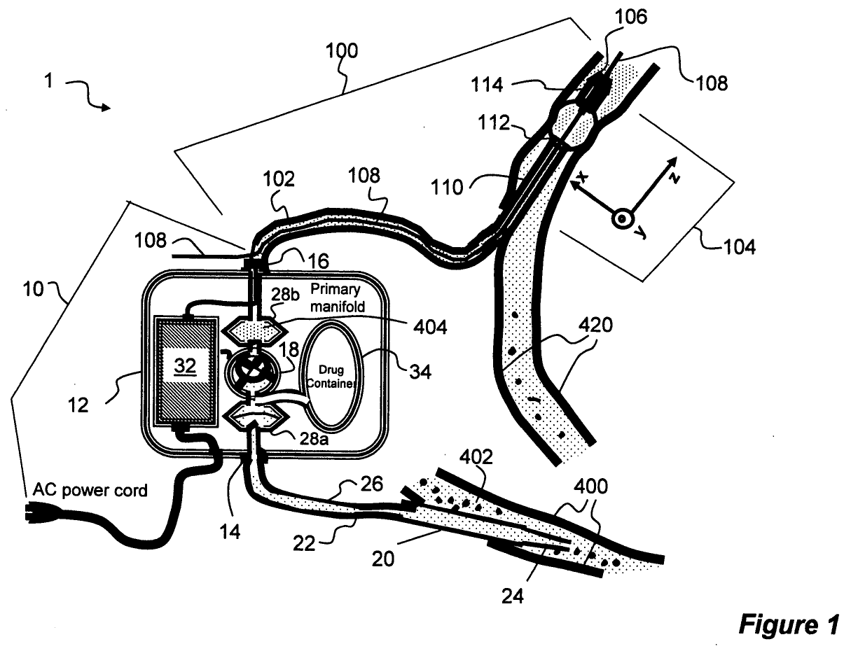

FIG. 1 illustrates an embodiment of the proposed blood deposits ablating apparatus 1 of the present invention as applied to a human or animal circulatory system to clean and remove plaques deposited on the inner lining of a blood vessel under treatment 420. The blood deposits ablating apparatus 1 includes a first blood extracting and pressurizing unit 10 for extracting source blood 402 from a supply blood vessel 400 supplying the blood, pressurizing the extracted blood into pressurized source blood 404 while processing it by, for example, filtering to remove any debris fragments that could clog up the blood stream, oxygenating it for a higher level of oxygen concentration or adjusting its temperature or PH value. The blood deposits ablating apparatus 1 also includes a second blood delivering and injecting unit 100. The blood extracting and pressurizing unit 10 includes a primary manifold 12 that further includes, but is not limited to, a primary inlet 14, a primary outlet 16 and a pumping device 18. In addition, the primary manifold 12 can also be equipped with an electrical subsystem 32 generating the many needed power and signals needed by the blood deposits ablating apparatus 1. For example, numerous DC power sources, numerous Radio Frequency (RF) power sources and a multi-frequency RF power signal generator might be included, etc. The primary manifold 12 can also include one or more primary blood buffers such as an aft chamber 28a and a fore chamber 28b in series connection with the pumping device 18. For those skilled in the art, each of the aft chamber 28a and fore chamber 28b can further include a one-way valve or valves to block an undesirable back flow of the extracted blood as shown. The aft chamber 28a and fore chamber 28b can be of stainless steel construction or they can be constructed with elastic materials such as fluorocarbon polymers, polyurethane or other elastomeric polymers that are pharmacologically inert. Using elastomeric chambers provides the added advantage in that, if their chamber body resonance frequency can be tuned to match the pulsing frequency of the pumping action, they can greatly enhance the percussion force of the blood injection into the blood vessel under treatment 420.

As an additional feature, the primary manifold 12 can be equipped with a drug container 34 communicatively connected to the primary blood buffers such as, in this case, the aft chamber 28a for supplying and metering an auxiliary drug at a pre-determined rate as desired by the blood deposits ablating apparatus 1 during its operation. For example, the auxiliary drug can be a solution for balancing the PH-value of the blood or an anticoagulant for blood clot prevention. Blood clot formation can be activated, as a result of the homeostasis reaction of the human or animal body, by a newly opened wound. For another example, the auxiliary drug can be a radio-contrast substance for injecting into the blood stream to enable the examination of the blood vessel under treatment 420 using X-rays. A third example is the oxygenation of the red blood cells. Other auxiliary drugs or medications can also be administered at the same time to increase the health benefit of the ablating process. These anticoagulant agents, radio-contrast substances or other additional optional drugs can be either premixed, or delivered through separate individually pressurized containers that are respectively metered, and injected into a Venturi tube (a cylindrical pipe with a constricted mid section) connecting the aft chamber 28a and the pumping device 18. The suction action of the pumping device 18, together with the center-section constriction of the Venturi tube, creates a pressure drop to pull the various drugs from their respective containers. Additionally, the drug container 34 and/or the other various containers can be made elastic and deformable to cause their contents automatically flow into the Venturi tube owing to the pressure drop at the center-section constriction, which in turn creates a pressure imbalance resulting in an inward movement of the container wall and gradually forcing the drug out of the container. While not shown here as an alternative, the drug container 34 can itself be equipped with its own supply micro pump and metering mechanism such as a solenoid-controlled needle valve. The parts of the drug metering needle valve are usually made of stainless steel, a pharmacologically inert polymer such as polyester, fluorocarbon polymer or a combination of these materials. Additionally, seals and 0-rings, made of various materials such as polyurethane, fluorocarbon polymers or other elastomeric materials, are employed in and around the valve.

Downstream of and in fluid-wise communicative connection with the blood extracting and pressurizing unit 10 is a blood delivering and injecting unit 100 for delivering and forcefully injecting the pressurized source blood 404 into the blood vessel under treatment 420. The blood delivering and injecting unit 100 includes a series connection of a flexible delivery tube 102, a secondary manifold 104 and an injector nozzle 106 that, upon its placement into a desired portion of the blood vessel under treatment 420, forcefully ejects the pressurized source blood 404 into the blood vessel under treatment 420 for ablating nearby plaques deposited along its interior surface. For piercing the blood vessel under treatment 420 and guiding the injector nozzle 106, the secondary manifold 104 and the delivery tube 102 along the blood vessel under treatment 420, the blood delivering and injecting unit 100 further includes a bendable guide wire 108 axially threaded through the delivery tube 102, the secondary manifold 104 and the injector nozzle 106. The secondary manifold 104 is made of a series connection of an upstream section of delivery catheter 110, at least one secondary storage chamber 112 for buffering the pressurized source blood 404 and a downstream section of injection catheter 114. For an added versatility, it is remarked that more than one secondary manifolds can be employed here with each associated secondary storage chamber connected to its own injecting nozzle. The secondary manifold 104 may also include additional functional devices such as an ultrasound transducer, etc. and these will be presently described. For convenience of illustration, the direction of blood flow within the blood vessel under treatment 420 is designated as Z-direction of a Cartesian coordinate system.

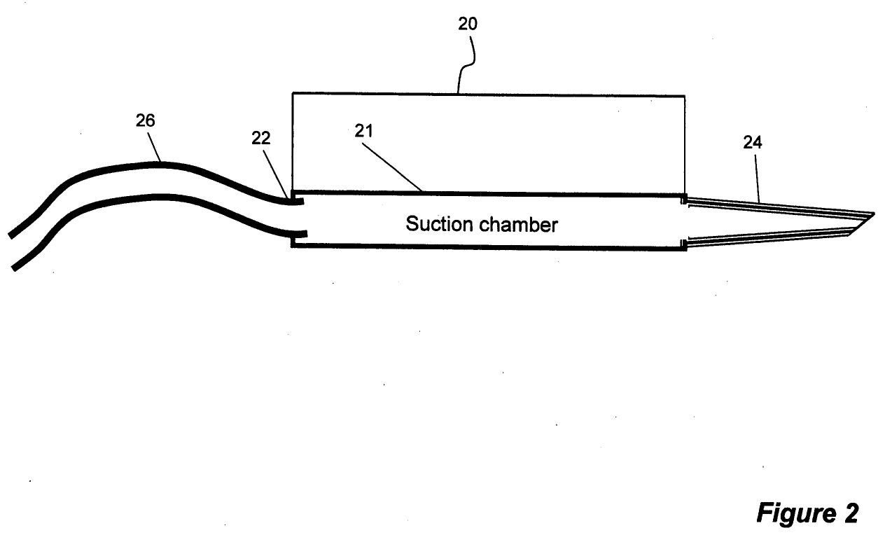

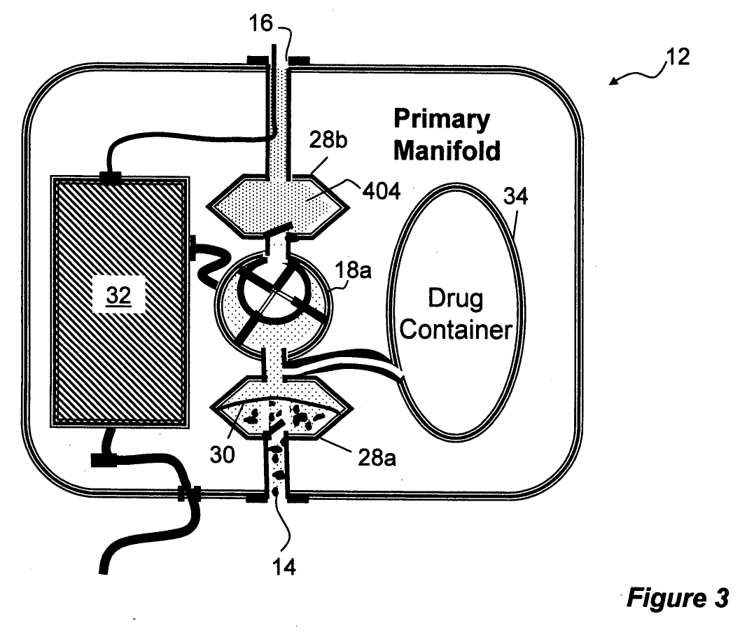

To facilitate blood extraction from the vein or artery of a human or an animal, the blood extracting and pressurizing unit 10 also includes a series connection of a hollow suction needle 24, a tertiary manifold 20 having a tertiary outlet 22 and a suction tube 26 delivering the extracted blood through the primary inlet 14. As part of the operation, the suction needle 24 is maneuvered to pierce the supply blood vessel 400 and draw blood from it. An enlarged illustration of the tertiary manifold 20 is shown in FIG. 2 having a suction chamber 21 located between the suction tube 26 and the suction needle 24 with a sharp tip that allows easy piercing through the skin and a blood vessel wall. Again for an added versatility, more than one suction needle 24 can be added to the tertiary manifold 20.

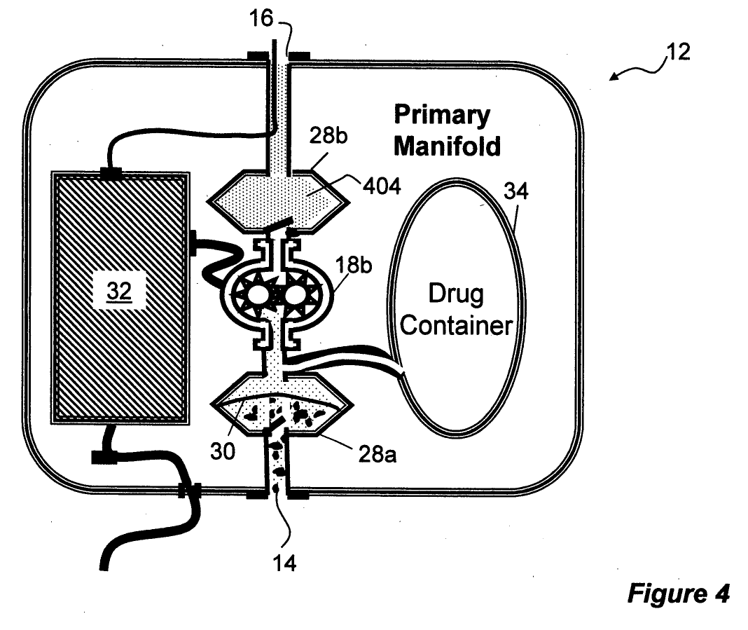

FIG. 3 illustrates an embodiment of the primary manifold 12 that, in addition to providing the functions of drug administration, charge signal and RF signal generation, uses a van pump 18a for blood pressurization and filtering. An inline blood filter 30 is added to the aft chamber 28a to filter out unwanted or undesirable substances including, among others, plaque fragments that exceed certain safety limit that could impede the blood flow. As already remarked before, both chambers 28a and 28b can also contain a one-way valve to insure that the blood can only flow in one direction. The drug container 34 stores auxiliary drugs that can be administered through a tube that is connected to the interconnecting pipe linking the outlet of the aft chamber 28a to the inlet of the van pump 18a. As the van pump 18a produces a suction pressure during operation, the drugs inside the drug container 34 will be administered automatically into the main blood circulation through this suction pressure. As remarked before, potential drug-specific benefits include coagulation prevention and rebalancing of the blood PH value. In addition, the drug container 34 may also contain a radio-contrast agent that absorbs X-rays for injection into the blood stream to enable the examination of the blood vessel under treatment 420 using X-rays as in X-ray angiogram or fluoroscopy. As an alternative, the same or a separate drug container can instead be included inside the secondary storage chamber 112 of the secondary manifold 104 and near the injector nozzle 106.

The electrical subsystem 32 is essentially a multiple output power supply that converts the mains (or other sources of electrical power) into various power sources of appropriate voltage or power and frequency for the blood deposits ablating apparatus 1. To begin with, the electrical subsystem 32 includes a low output impedance Direct Current (DC) power source to drive the van pump 18a. Another power source, called ultrasonic power supply, is a high frequency power electrical signal at least 100 KHz (Kilo Hertz) in frequency, more preferably in the 1-10 MHz (Mega Hertz) range, and with a power rating ranges from 1Watt to 200 Watt. As will be presently described, this ultrasonic power supply is primarily used to drive an ultrasonic power transducer for the pulverization and emulsification of undesirable deposits and diseased blood tissue inside the blood vessel under treatment 420. In a preferred embodiment, the ultrasonic power supply includes two or more frequency components that are close in frequency values. Under a dual-usage concept with proper power signal routing and switching, the same ultrasonic power supply can also be used to create an RF discharge in the blood stream near the injector nozzle 106 hence administering a localized intense RF heating to ablate through severe plaque blockages or to destroy diseased tissue during the ablating process. The ultrasonic pulverization and emulsification and localized RF heating thus function to at least complement the abatement of deposited plaques with pressurized source blood injection and, for mid to late stage atherosclerosis wherein the deposited plaques can be hardened and thick, can function as the dominant mode of treatment. In addition, the electrical subsystem 32 also includes a high output impedance DC power source for delivery to a DC discharging tip near the injector nozzle 106 thus supplying positive charges to neutralize excess negative charges generated from the tearing of healthy or diseased tissues during the ablating process. Of course, in cases where excess positive charges are generated during the ablating process, negative charges should be supplied from the DC discharging tip instead. The essence is that electrical neutrality should represent the most stable biomedical state. Accordingly, the electrical subsystem 32 includes an electrical discharge supply circuit having the high impedance DC power source as its output. Yet another power source provided by the electrical subsystem 32 is a low power RF source, for ultrasonically illuminating hence intravascular ultrasound imaging of the blood vessel interior under treatment, with an imaging frequency in the range of 10 MHz to 100 MHz and power rating of less than about The ultrasonic illumination and image detection can be accomplished with a low power imaging frequency ultrasonic transmitter, driven by the low power RF source, located near the injector nozzle 106.1 Watt. Accordingly, the electrical subsystem 32 includes an imaging frequency signal generator having the low power RF source as its output. For those skilled in the art, the multiple signal and power outputs from the electrical subsystem 32 are isolated from one another and further isolated from the mains for safety of the patient and personnel involved with the ablating process. While not shown here to avoid unnecessary obscuring details, the multiple signal and power outputs are delivered to their final destination of usage, the secondary manifold 104, through a multi-conductor thin coaxial RF waveguide cable which also carries DC current. Of course, the RF waveguide cable would need to thread through the primary outlet 16, the delivery tube 102 and the secondary manifold 104.

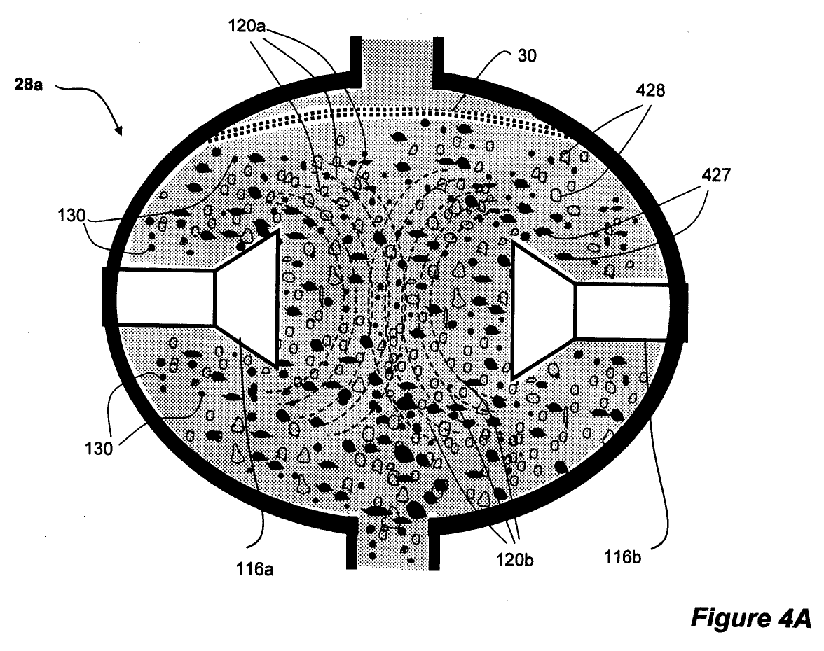

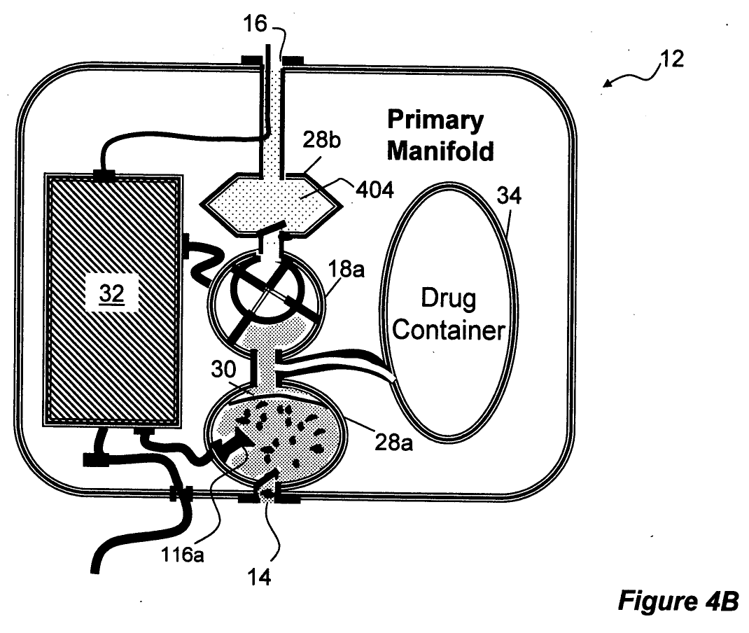

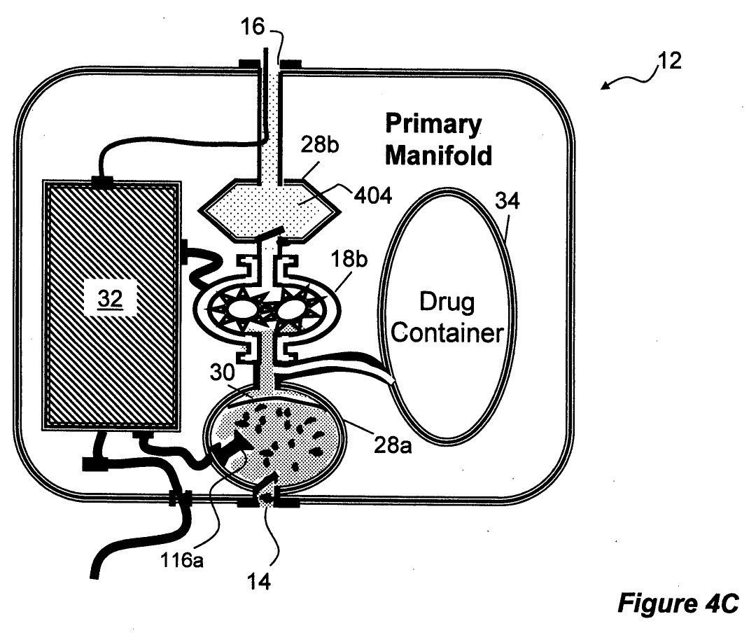

FIG. 4 illustrates another embodiment of the primary manifold 12 that, otherwise the same as shown in FIG. 3, uses a gear pump 18b instead for blood pressurization and filtering. Other types of fluid pumping devices such as lobe pump, peristaltic pump and centrifugal pump, etc. can also be used as well.

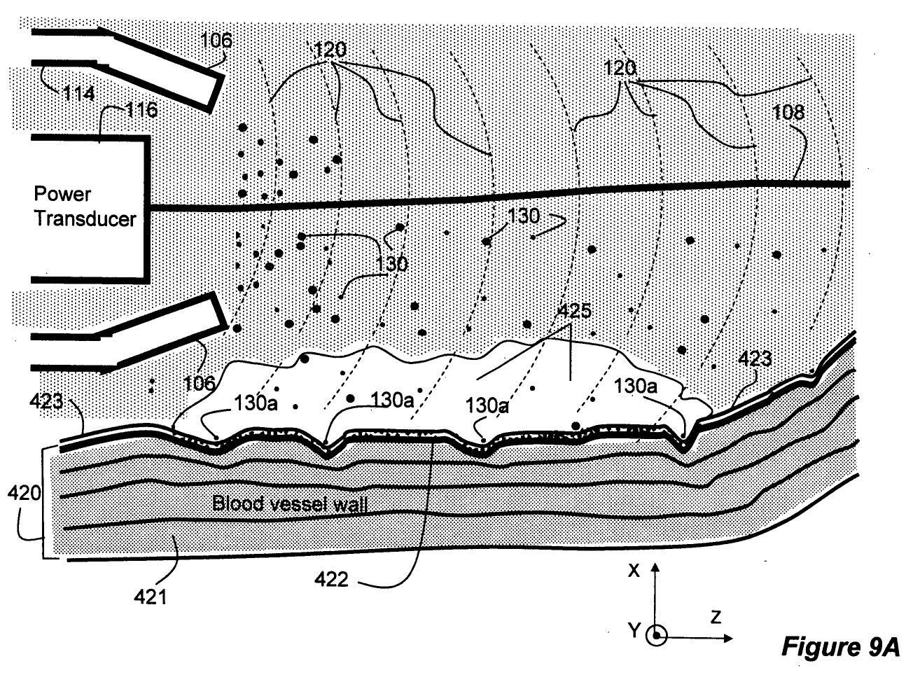

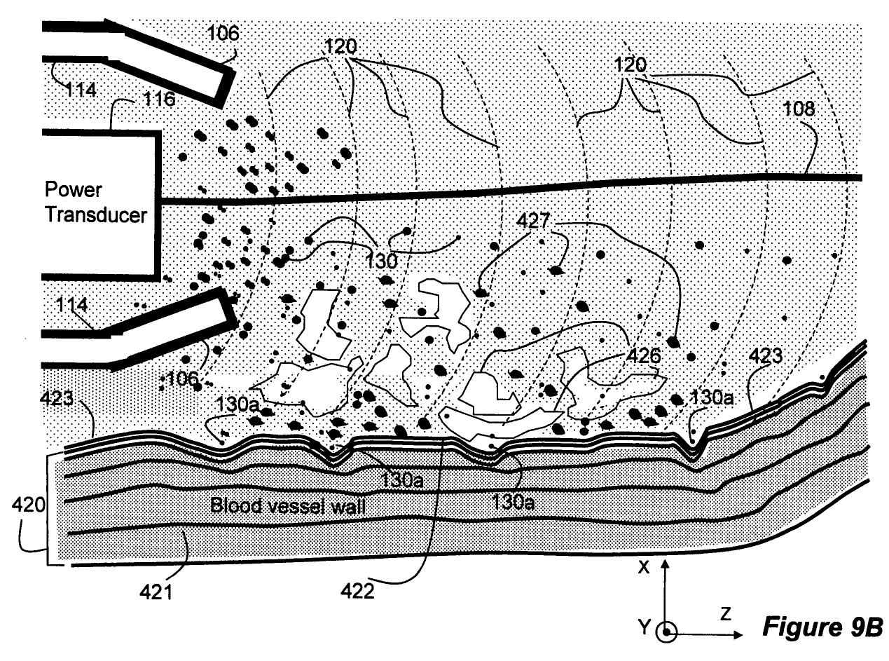

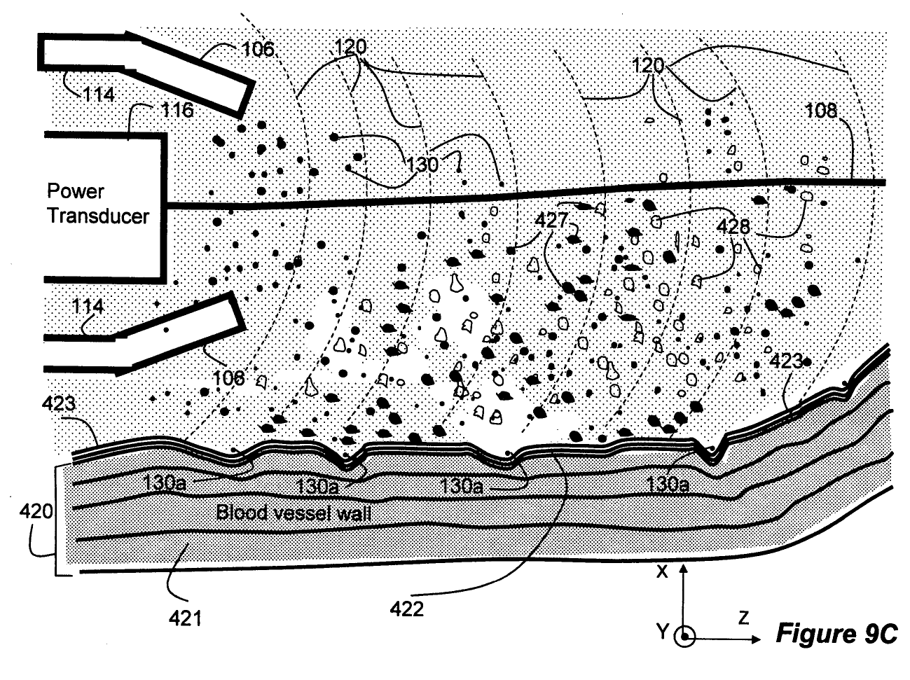

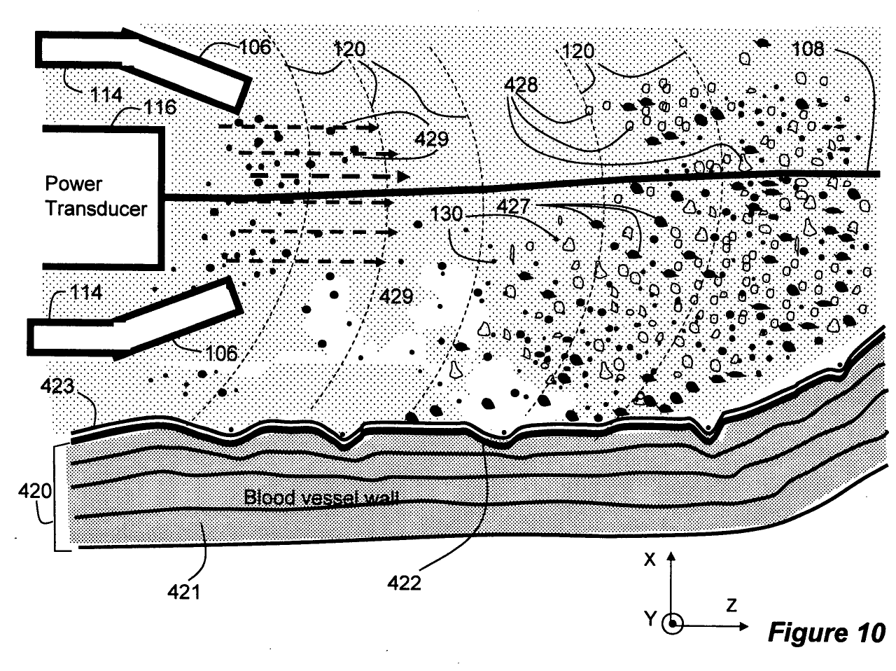

FIG. 4A illustrates an improvement of the blood filtration inside the aft chamber 28a of the primary manifold 12 using power transducers 116a and 116b located upstream of the inline filter 30 for respectively emitting ultrasonic power emissions 120a and 120b into the blood to pulverize and emulsify, via ultrasound induced cavitations 130, undesirable substances of the extracted source blood into microscopic calcified fragments 427 and microscopic plaque fragments 428. The underlying physics of the ultrasonic cavitation process together with its power to pulverize and emulsify certain undesirable substances within the blood will be presently described from FIG. 9A through FIG. 10. In essence, the thus improved aft chamber 28a acts as an ultrasound emulsification chamber. As pulverization and emulsification greatly reduce the size of these undesirable substances, they reduce the corresponding particulate loading upon the inline filter 30 hence enhancing its effectiveness. As the aft chamber 28a is located outside a blood vessel, the size and shape of the aft chamber 28a can be specially tailored to produce a strong resonant standing wave of ultrasonic power emissions 120a and 120b. Furthermore, the temperature of the aft chamber 28a can be maintained within a pre-determined range so that it is conducive to the generation of intense ultrasonic cavitations. A preferred embodiment of the temperature is estimated to be in the range of about 60° C. to about 80° C. Since this external ultrasound emulsification chamber can be optimized for breaking down large grain sized debris, the aft chamber 28a can be made far more effective in emulsifying the plaque and calcification fragments than its in vivo counterpart. FIG. 4B is simply an overview of the primary manifold 12 embodied with the above improvement of the blood filtration using power transducer 116a in combination with a van pump 18a. Similarly, FIG. 4C is an overview of the primary manifold 12 embodied with the above improvement of the blood filtration using power transducer 116a in combination with a gear pump 18b.

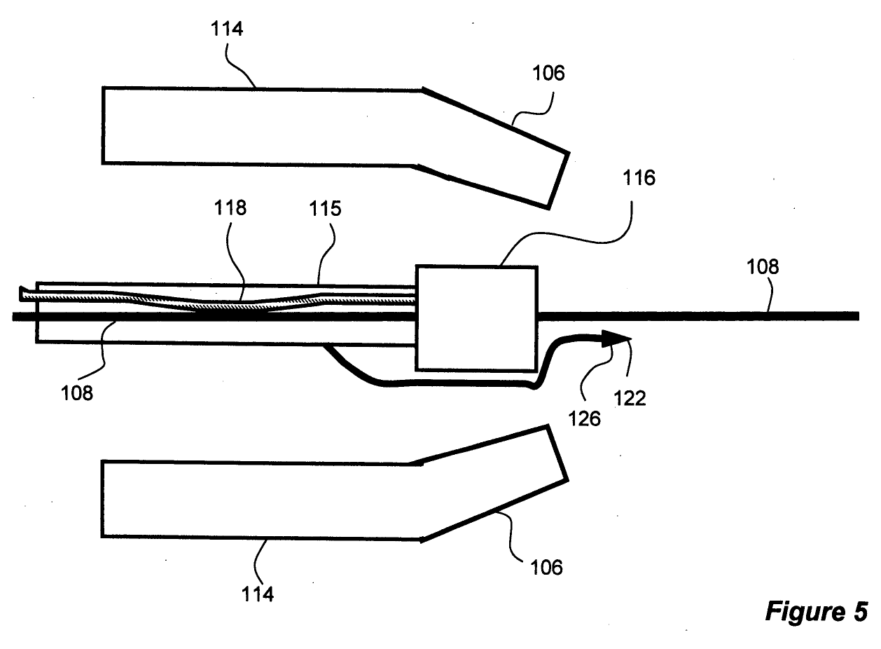

FIG. 5 illustrates the front end of the blood delivering and injecting unit 100 of the proposed apparatus that includes a guide wired injector head with an injector nozzle 106. In this embodiment, the front end of the blood delivering and injecting unit 100 is constrained and guided by the guide wire 108 which is maneuvered to pierce and thread through the blood vessel under treatment 420 first. The outer substantially cylindrical injection catheter 114 ends with a convergent structure forming the injector nozzle 106. An inner tube 115 ends with an attached ultrasonic power transducer 116 for the pulverization and emulsification of undesirable deposits and diseased blood tissue inside the blood vessel under treatment 420. This is accomplished with an ultrasonic power emission from the power transducer 116 during operation causing cavitation in the blood.

Another attachment to the end of the inner tube 115 is a DC discharging tip 122, located near the injector nozzle 106, for supplying charges to neutralize excess opposite-sign charges generated from the tearing of healthy or diseased tissues during the ablation process as mentioned before. Without impairing its intended functionality, the DC discharging tip 122 can be made as part of the injector nozzle 106 as well. While not shown here to avoid obscuring details, the DC discharging tip 122 can be powered by a high output impedance DC power source located either within the injection catheter 114 or within the electrical subsystem 32. The DC discharging tip 122 could be made of a thin wire with a typical diameter ranging from 0.004 inch to 0.012 inch, although either smaller or larger diameters are also acceptable. The wire material is preferably stainless steel or gold. Alternatively, the DC discharging tip 122 could be a needle or an array of needles made of stainless steel or gold. In order to prevent the known physical phenomenon of Debye shielding by opposite-sign ions in the blood, deionized water can be co-injected with the supply of neutralizing charges. By surrounding the DC discharging tip 122 with deionized water, the Debye shielding effect can be neutralized so that the charges emitted from the DC discharging tip 122 can be delivered to the diseased area of the blood vessel under treatment 420. Yet another attachment to the end of the inner tube 115 is a heating device in the form of an RF discharging tip 126 for creating the RF discharge in the blood stream as mentioned before. Physically, the RF discharging tip 126 can simply be the same discharging tip used for the DC discharging tip 122, or the RF discharging tip 126 could be made of a separate needle that further contains a bundle of curved, retractable antennas (thin wires made of stainless steel or gold) that are kept inside the needle until its tip gets positioned within a treatment area. In cases where 60 GHz (Gegahertz, 109 Hz) or higher RF frequencies are employed, the RF discharging tip 126 can be alternatively implemented with a directionally steerable antenna such as a micro-dish antenna of around 4 mm in diameter or, preferably, an electronically steerable phased array antenna of like dimensions. The ability of the steerable antenna to direct and focus the RF energy to where it is needed is an important benefit of the millimeter wave (RF with wavelength in the millimeter range) technology. Of course, the heating device can have numerous alternative forms of implementation, other than the RF discharging tip 126, such as a resistive heater, a thermal electric device or a magnetic induction heater. Also, without impairing its intended functionality, the heating device can be made as part of the injector nozzle 106 as well.

To avoid unnecessary obscuring details, yet another attachment to the end of the inner tube 115, a low power imaging frequency ultrasonic transmitter for. intravascular ultrasound imaging of the blood vessel interior under treatment, is not graphically illustrated here. The ultrasound imaging device includes a steerable phased array ultrasound transceiver. The ultrasound transducer emits a directional ultrasound beam that revolves at high-speed, of the order of 1800 rpm or 3600 rpm, to provide a 30 or 60 frames per second temporal resolution of a 360° real-time video image of the blood vessel interior under treatment. Alternatively, a separate single transducer plus a rotating receiver revolving at around 1800 rpm can also create a 3600 video image perpendicular to the Z-direction with lateral and axial resolutions of roughly 150 micron (10⁻⁶ meter) and 90 micron, respectively with a 30 MHz ultrasound transducer frequency. With a higher ultrasound transducer frequency, the spatial image resolution correspondingly increases.

As a selective illustration, a high frequency power electrical signal 118 for driving the power transducer 116 is shown. For material isolation from the blood and electrical insulation, the guide wire 108 as well as the multi-conductor thin coaxial RF waveguide cable are enclosed within the inner tube 115.

FIG. 6 illustrates, with a side view and a three-dimensional perspective view, an embodiment of the power transducer 116 head with its driving high frequency power electrical signal 118 delivered through a waveguide structure. Notice that the front face of the power transducer 116 is designed with a multiple concentric ring.

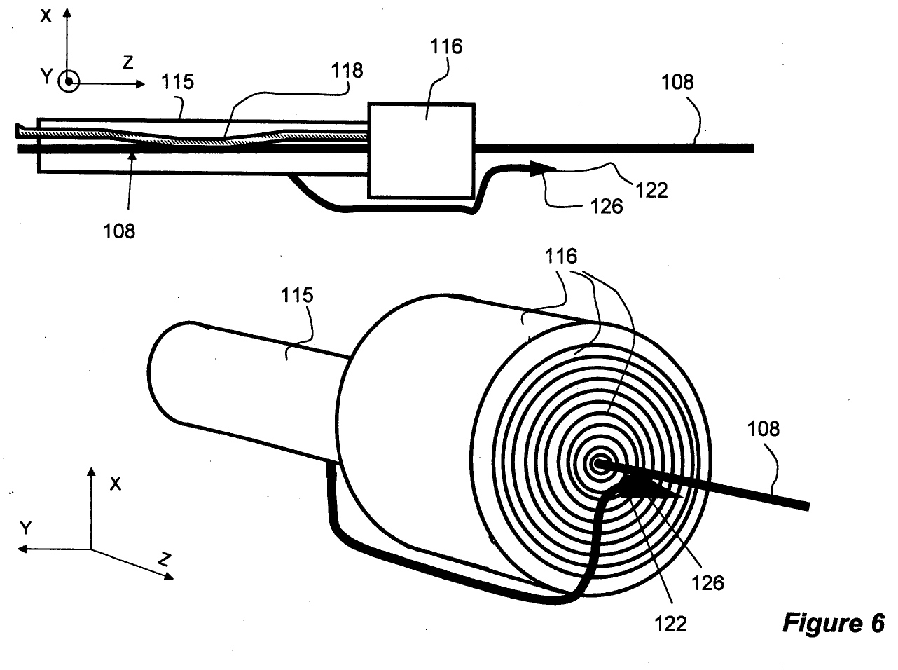

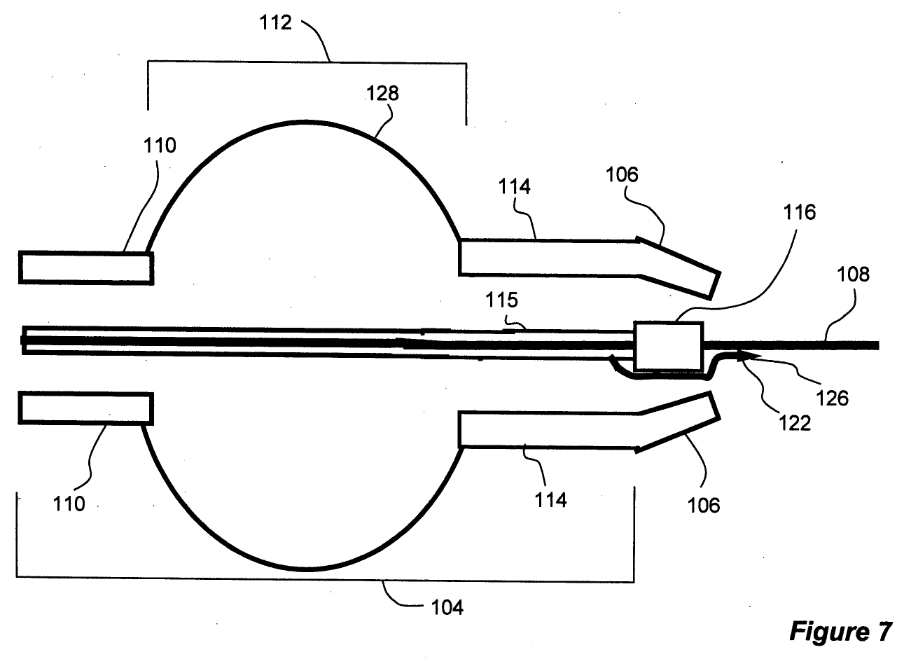

FIG. 7 illustrates more embodiment of the secondary manifold 104 having a foldable isolation balloon 128. The interconnecting electrical power and signal lines are not shown to avoid unnecessary obscuring details. Being located just upstream of the injector nozzle 106, the inflated foldable isolation balloon 128 substantially blocks the lumen of the blood vessel under treatment 420 and serves to isolate the localized elevated blood pressure hence maintaining a pressure difference and preventing the injected pressurized blood from flowing in the backward direction (-Z direction). The inflated foldable isolation balloon 128 also serves to anchor the secondary manifold 104 against the blood vessel under treatment 420 to absorb a reaction force produced by the forceful injection of the pressurized blood and a radiation force produced by the ultrasonic power emission pulses. Simultaneously, with the foldable isolation balloon 128 inflated within a safety stretch limit by the properly driven pumping device 18, the inflated foldable isolation balloon 128 gets pushed along in the Z-direction under the same pumping action.

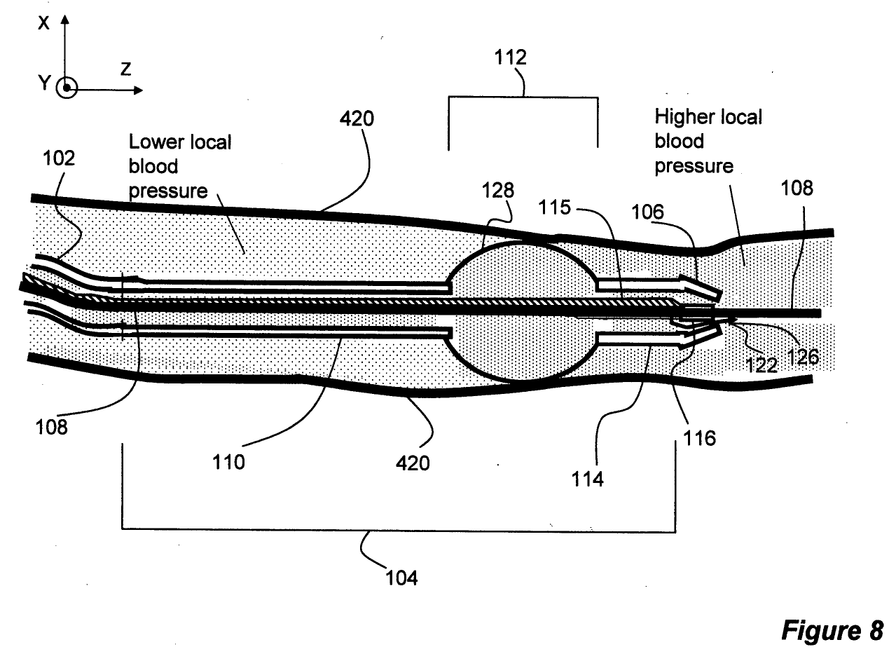

FIG. 8 further illustrates the placement of the injector nozzle 106 and the secondary manifold 104 having the foldable isolation balloon 128 inside the blood vessel under treatment 420 during an ablating procedure. In this case, the blood vessel under treatment 420 could be an artery. First, the guide wire 108 is threaded through the blood vessel under treatment 420. The secondary manifold 104 is then slowly advanced through the artery to a diseased area while the foldable isolation balloon is kept deflated and folded. Once the secondary manifold 104 is positioned and properly aligned, the pumping device 18 within the primary manifold 12 is activated to supply filtered and pressurized blood to the secondary manifold 104 through the delivery tube 102. The continued pumping action causes the pressurized blood to be forcefully injected through the injector nozzle 106 onto a diseased region just downstream of the injector nozzle 106. This creates a pressure drop across the injector nozzle 106 which in turns causes the foldable isolation balloon 128 to inflate until it touches and slightly expands the artery wall within a safety limit, thereby blocking the artery passage and preventing the injected pressurized blood to flow backward around the foldable isolation balloon 128 to the upstream side. Now the power transducer 116 and the RF discharging tip 126 are energized to ablate, through pulverization with the accompanying ultrasonic power emission, plaques that are directly downstream of the injector nozzle 106. Subsequently, the pulverized plaque fragments are further emulsified by the mixing action of the ultrasound induced turbulent flow and then pushed downstream by the forceful blood injection as well as by the radiation pressure of the high power ultrasonic wave itself. Simultaneously, the DC discharging tip 122 is energized to discharge charges into the blood to neutralize excess opposite-sign charges generated by the tearing of the plaque tissue from the otherwise healthy, smooth muscle tissue on the artery wall. Concurrently, the power transducer 116 can also be energized with the imaging frequency to emit a higher imaging frequency ultrasound for intravascular imaging.

As mentioned before, the ablation of deposited plaques by the ultrasonic power emission is based upon the formation cavitations. More specifically, an intense ultrasound wave with wavelengths smaller but not substantially smaller than the inside diameter of the blood vessel wall will create a multitude of micro cavities, each being a partial vacuum, in a fluid that collapse rapidly with an implosion. The mechanical energy released by the sudden “implosion” is responsible for its ability to pulverize hardened calcification layer under the diseased tissue. These micro cavitations are quite small, of the order of micrometers in diameter when they collapse. The imploding cavitations work best in attacking hard, fragile and inelastic substances such as a calcified tissue. The cavitations will break up the soft, albeit inelastic diseased tissue of a plaque as well. On the other hand, they will have virtually no effect on otherwise flexible and highly elastic healthy muscle tissue that form the bulk of the blood vessel wall as the collapsing cavities can only provide primarily highly localized mechanical bending and compression but no tearing action. The flexibility and elasticity of the healthy tissue can easily absorb the imploding pressure with a slight local deflection and/or compression. But a hardened calcified tissue is too stiff to yield to the bending and compression stress of the localized implosion hence will be shattered by it.

Furthermore, as the calcified tissue is rigid enough to support one or more mechanical vibrational resonances, it should be possible to periodically vary the frequency of the ultrasonic power emission so as to tune it to the various mechanical resonances of the calcified tissue thus further enhancing the ability of the ultrasound to shatter and pulverize the calcified tissue. Because of the small size of the collapsing cavities, they can pulverize the plagues into correspondingly small debris particles no more than a few micrometers in diameter for safe passage through the arteries with virtually no embolus. On the other hand, as the location of cavitations typically concentrates around a nodal point of the ultrasound standing wave, to ensure a spatially uniform pulverization of the plagues the coordinates of the nodal points should be made time dependent. This can be achieved through the simultaneous use of two or more ultrasound components close in frequency to produce a spatially slowly varying standing wave pattern.

As a high volumetric energy density is needed to generate cavitation, the ultrasonic power emission should be spatially confined to the lumen (the interior opening of the blood vessel) by reflection to be effective. As the mass density of the blood vessel wall is not much higher than that of the blood itself, an effective way to make the ultrasound reflect from the vessel wall is to use an ultrasound wavelength that is smaller than the vessel wall thickness. At longer wavelengths, the ultrasound would simply locally enlarge the vessel where the local pressure is high and shrink it where the local pressure is low. Very little bending of the vessel wall is produced when the wavelength is much larger than the wall thickness. However, when the ultrasound wavelength becomes much shorter than the wall thickness, the positive and negative pressure regions are now located close together on the inner wall surface and only local wall deformations are formed. Furthermore, these deformations do not extend much beyond a wavelength into the wall thickness, therefore the wall now appears to be rigid and reflection of the ultrasound wave results. Hence the confinement of the ultrasound energy is better with a shorter wavelength ultrasound wave. On the other hand, under the same power level of the ultrasonic power emission, cavitation typically increases with the ultrasonic wavelength. To balance these two opposing mechanisms, the ultrasonic wavelength λ should be set as approximately the geometric mean, defined as the square root of the product, between the vessel wall thickness T and the diameter of the vessel lumen D. Or, mathematically:
λ=square root (T×D) (1)
In this way, while the geometric mean is larger than the wall thickness, it is not overly so, hence the resulting ultrasound confinement is still good at such a wavelength. Meanwhile, λ is also not much smaller than the lumen diameter D thus allowing a well defined standing wave pattern to be established in the radial direction (perpendicular to the Z-direction), a condition that further favors the formation of strong cavitations. As a quantitative example, the speed of sound propagation within the blood stream is about 1400 meters per.second. The artery diameter of a typical human being is approximately 3 mm to 6 mm, hence the calculated ultrasound frequency should be in the 0.5 MHz (MegaHertz) to 2 MHz range to obtain a good ultrasound confinement. For small animals, the ultrasound frequency should be higher than while for large animals the ultrasound frequency can be smaller than the aforementioned values. Once the calcified deposit has been pulverized into small particles, the turbulent blood motion resulting from the combined effect of pressurized blood injection and the high power ultrasound works to thoroughly mix the pulverized particles, thus emulsifying them with the blood and thereafter propelling them downstream.

FIG. 9A illustrates an ultrasonic cavitation process together with its initial interaction with a diseased plaque tissue 425 located along the inner intima lining 423 of the atherosclerotic blood vessel under treatment 420, in this case an artery, during the ablating process. As shown, the cavitations 130 are formed near the nodal points of the partial standing wave resulting from multiple reflections of the ultrasonic power emission 120 from the blood vessel wall whose elasticity mainly comes from the smooth muscle 421. Upon collapsing, each of the cavitations 130 implodes violently causing an intense localized pressure that resonates with the diseased plaque tissue 425. Those surface cavitations 130a, the ones collapsing around the wall, cause sharp local bending and compression of those hardened inner surfaces such as the diseased plaque tissue 425. The diseased plaque tissue 425 includes a loose, foamy collection of fatty deposits together with a fibrous cover (or cap) that are attached to the inner intima lining 423. This plaque material is soft and fragile with little elasticity. As the artery is contracted and expanded with each beating of the heart, the adhesion between the plaque and the inner intima lining 423 gets loosened. This in turn allows Calcium deposit to accumulate in the gap between the outer portion of the plaque and the muscular blood vessel wall, forming a calcified layer 422. Such calcification process will gradually progress with time and ultimately lead to a loss of elasticity and stiffening of the artery as a whole.

FIG. 9B illustrates a mid stage interaction between the cavitations 130, the surface cavitations 130a and the diseased plaque tissue 425 during the ablating process. By now the resonating diseased plaque tissue 425 and its underlying calcified layer 422 are shattered into broken-up plaque fragments 426. Ultrasound induced cavitations 130 and 130a further resonate, bend and disintegrate the broken-up plaque fragments 426 and calcification into small, sharp microscopic calcified fragments 427 that regeneratively dislodge or perforate the diseased plaque tissue 425 and its covering fibrous tissues.

FIG. 9C illustrates a late stage interaction between the ultrasonic cavitation and the diseased plaque tissue 425 during the ablating process. The soft diseased plaque tissue 425 and its fibrous covering are finally broken up and pulverized by the vigorous movement of the microscopic calcified fragments 427 driven by the ultrasound induced random implosions of the cavitations 130. Each implosive event accompanying the cavitations 130 sends a nearby dense calcified fragment flying in a ballistic fashion. Multitude of bullet-like calcification fragments puncture and grind the soft, inelastic, fragile broken-up plaque fragments 426 into microscopic plaque fragments 428 that are thoroughly mixed with the blood into an emulsion for a safe passage downstream.

By now it should become clear that, when properly used, the ultrasonic power emission should be a safe and effective way to remove plaques and concomitant calcifications in atherosclerosis. Ultrasound induced cavitations can differentially pulverize the dense calcification deposits and then set the pulverized hard calcification fragments in vigorous ballistic motions. In turn, such motions of the hard calcification fragments can easily cut and perforate the soft, foamy yet inelastic plaque tissue and its associated fibrous cap without hurting the nearby healthy muscular tissue that constitutes the blood vessel wall. Upon complete removal of the calcification deposits from the diseased area, the ultrasound ablating action terminates automatically as the ultrasound can not harm the elastic healthy tissues. Therefore, it should be possible to employ the proposed blood deposits ablating apparatus 1 to safely clean and remove diseased deposits even during an early stage of the plaque formation when there is either no or otherwise insignificant protrusion of plaque growth into the lumen. Additionally, the frequency scanning aspect of the proposed blood deposits ablating apparatus 1 further allows a lower level ,of ultrasound power to be used for such a procedure by taking advantage of resonant shattering of the calcification substances.

FIG. 10 illustrates the excavation of post-pulverization plaques and calcified debris away from a diseased area of the blood vessel under treatment 420. The excavation action is accomplished by the forceful injection, from the injector nozzle 106, of pressurized blood as well as by a radiation pressure created by the ultrasonic power emission 120. As illustrated, a pressurized blood flow 429 results from the forceful injection of pressurized blood. Hence, the plaque debris gets pushed forcefully downstream and away from the diseased region. In a preferred embodiment, the suction needle 24 should be inserted into a nearby downstream location of the same blood vessel under treatment 420 if possible to facilitate the collection, via the inline filter 30, of the microscopic calcified fragments 427 and microscopic plaque fragments 428. Additionally, the tertiary manifold 20 can be further provided with one or more optional power transducers, affixed in proximity to the tip of the suction needle 24, for emitting corresponding ultrasonic power emissions into the blood to remove the undesirable deposits inside the blood vessel under treatment 420 via multi-staged pulverization and emulsification. For those skilled in the art, by now it should also become clear that the blood deposits ablating apparatus 1 can be bi-directionally operated in that the injection catheter 114 can either be pushed along in the Z-direction or pulled backwards, after reaching a pre-determined depth location within the blood vessel under treatment 420, in the negative Z-direction during the ablating process.

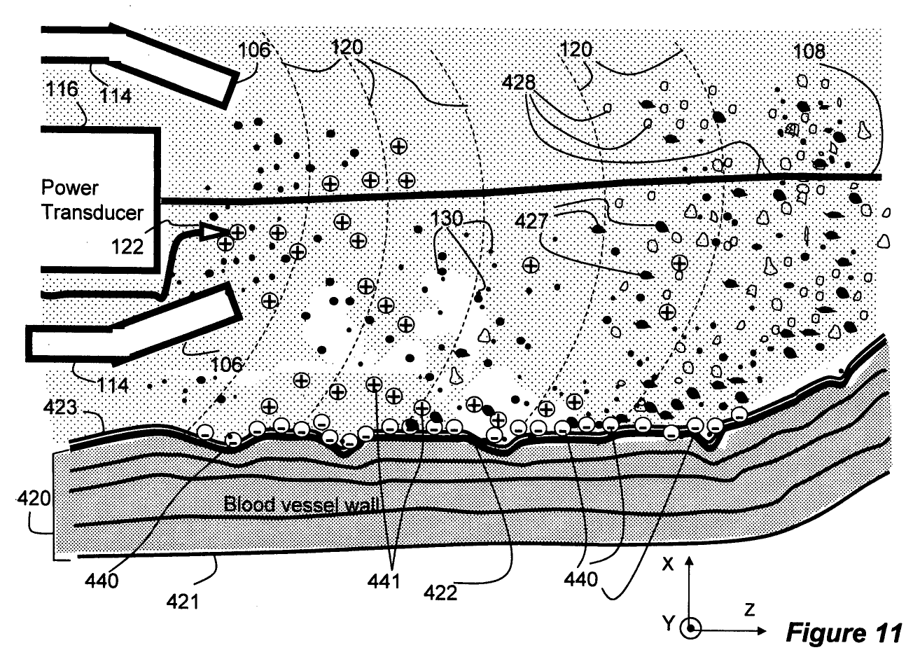

FIG. 11 illustrates the neutralization of, as an example, negative surface charges 440 atop a newly formed tissue wound by positive charge emission from a DC discharging tip 122 located inside the injector nozzle 106. With the DC discharging tip 122 simultaneously energized by the electrical subsystem 32 during the ablating process, the negative surface charges 440 that populate the newly formed tissue wound are neutralized by positive space charges 441 emitted by the DC discharging tip 122. The neutralization of excess surface charge buildup can reduce blood coagulation and can promote healing of the wounded tissues beneath the undesirable deposits. Recalling from FIG. 8 that the outside surface of the foldable isolation balloon 128 is located very close to the inner wall of the blood vessel under treatment 420 with an annular portion in actual physical contact with the wall, the DC discharging tip 122 can alternatively be distributed around the outside surface of the foldable isolation balloon 128 for providing discharges in close proximity or in direct contact with the diseased or torn healthy tissues to neutralize the generated excess charges with better efficiency.

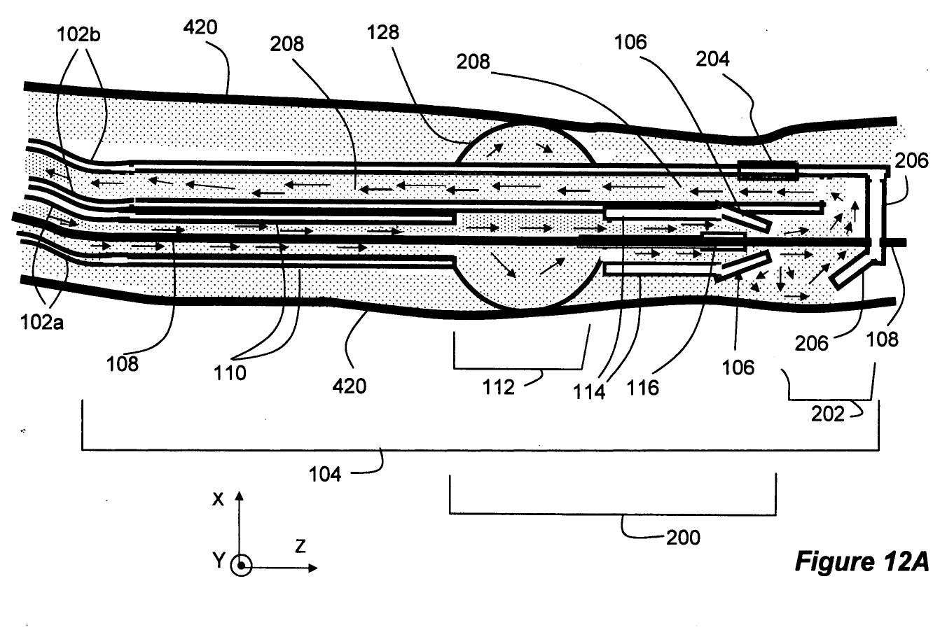

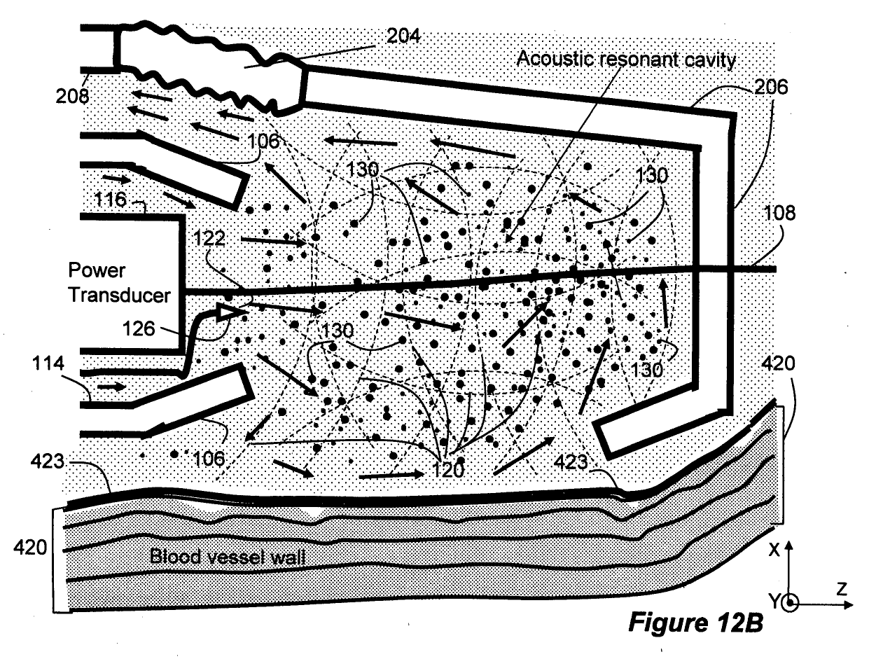

FIG. 12A and FIG. 12B together illustrate a further improvement of the present invention using a dual tube concept with an end ultrasonic cavity as the front portion of the blood delivering and injecting unit. FIG. 12A shows another embodiment of the present invention wherein the tertiary manifold 20 is now combined with the secondary manifold 104 (see FIG. 1) into a single manifold that includes an injection and ablation unit 200 and a reception and confinement unit 202 inter-connected with a semi-flexible interconnect tube 204, with the reception and confinement unit 202 located only slightly downstream of, for example a few millimeters away from, the injection and ablation unit 200. For clarity, the injection and ablation unit 200 includes the injector nozzle 106, the power transducer 116, the DC discharging tip 122, the RF discharging tip 126, the injection catheter 114 and the secondary storage chamber 112 with the foldable isolation balloon 128 in this embodiment. The reception and confinement unit 202 includes a deflector head 206, the semi-flexible interconnect tube 204 and a receptor tube 208. The deflector head 206 serves to confine and actively collect plaque and calcification debris by suction. The receptor tube 208 routs the debris laden blood for returning to the primary manifold 12 through its primary inlet 14 for the removal of the debris by the inline filter 30 as well as for additional blood conditioning via the drug container 34. As the now improved secondary manifold 104 advances through the blood vessel under treatment 420, both the injection and ablation unit 200 and the reception and confinement unit 202 move in unison with the semi-flexible interconnect tube 204 providing a needed range of flexible movement between them for negotiating numerous bends of the vessel wall as it wend its way along the body interior. With this arrangement, the potentially blood clogging debris never travels far before it gets collected. Therefore, even if the blood deposits ablating apparatus 1 should accidentally produce some fragments that are larger than 5 micron (1 micron=10⁻⁶ meter), or roughly the size of a red blood cell, this is inconsequential as the fragments do not have a chance to travel downstream into the blood circulation system to cause any potential damage.

FIG. 11B shows that the reception and confinement unit 202 further serves to reflect and confine the ultrasonic power emission 120 emitted from the power transducer 116 of the injection and ablation unit 200. Together with the reflection of the ultrasound from the inner intima lining 423, the reception and confinement unit 202 forms a sort of acoustic wave guide having a number of resonant frequencies. As the ultrasound frequency can be varied in time with the electrical subsystem 32, the ultrasound frequency will sweep through the above resonant frequencies with the consequence that the ultrasound wave amplitude will drastically increase at these resonant frequencies. This in turn causes a drastic increase in the strength of cavitations 130 hence their ablation power. Even if the ultrasound frequency is not near one of these resonant frequencies, the very fact that the ultrasonic power emission 120 is being reflected back to the acoustic wave guide means that the ultrasound energy density in the confined region increases as the energy leakage of the ultrasound wave is minimal. Meanwhile, the time varying acoustic radiation pressure of the focused ultrasound wave inside the acoustic cavity also creates strong agitating force and further enhances the turbulent mixing of the blood emulsion. By reducing the ultrasound leakage with the reception and confinement unit 202, another benefit of the confinement effect is that it can help limit any potentially negative biological effect of the high power ultrasound wave on otherwise healthy tissues away from the diseased region under treatment.

Yet another benefit of this embodiment is the reduction of discomfort a patient might otherwise experience as only one point of invasion into the patient's body is needed here. Additionally, the proposed embodiment automatically collects and can recycle drugs that had been administered by the injection and ablation unit 200. This can be beneficial in that certain kinds of drugs primarily designed for treating the diseased area but may otherwise be too toxic and potentially dangerous if they were leaked into the healthy areas. Hence, a wider variety of drugs can be used in a more targeted fashion.

When used in conjunction with RF ablation by the RF discharging tip 126, the needs to efficiently focus and concentrate the RF power is similar to the case of ultrasound ablation. To effectively reflect an RF field, however, the deflector head 206 needs to be made conductive. This can be easily satisfied with a metallic construction of the deflector head 206, or alternatively a non-metallic deflector head 206 with a conductive surface coating.

The dual tube arrangement as described above, having a delivery tube 102a connected to an injection and ablation unit 200, and a receptor tube 208 connected to a reception and confinement unit 202, further makes it possible to discharge positive charges in a bipolar, instead of a unipolar fashion. With a unipolar discharge scheme, only charges of one polarity are delivered to the destination while the charges of the other polarity are simply grounded. Within an ionized fluid, such as blood, such a unipolar discharge scheme may be hindered by a phenomenon called Debye shielding where the discharge electrode gets electrostatically shielded by ions of opposite charge in the blood, thus rendering the discharging action less effective. However, with a bipolar discharge scheme, charges of both polarities are delivered in equal amount to the destination. Specifically, the reception and confinement unit 202, now intentionally made electrically conductive, works as the cathode that can draw the positive charges, in the form of positive ions, from the DC discharging tip 122. With proper geometric shaping of the cathode, the positively charged ions can then be made to contact the excess negative surface charges 440 on the diseased surface to neutralize them hence enhancing the efficiency of charge neutralization.

As described with numerous exemplary embodiments, an apparatus is proposed for ablating undesirable deposits along the inner linings of blood vessel walls of human and animals. However, for those skilled in this field, these exemplary embodiments can be easily adapted and modified to suit additional applications other than those associated with blood vessels of human and animals without departing from the spirit and scope of this invention. Thus, it is to be understood that the scope of the invention is not limited to the disclosed embodiments. On the contrary, it is intended to cover various modifications and similar arrangements based upon the same operating principle. The scope of the claims, therefore, should be accorded the broadest interpretations so as to encompass all such modifications and similar arrangements.

We claim:

1. An apparatus for ablating undesirable deposits along the inner blood vessel wall of human and animals, the apparatus comprising:
a blood extracting and pressurizing unit for extracting source blood from a supply blood vessel and pressurizing the extracted source blood; and
a blood delivering and injecting unit, in communicative connection with said blood extracting and pressurizing unit, for delivering and forcefully injecting the pressurized source blood into a blood vessel under treatment, wherein the direction of blood flow is designated as Z-direction of a Cartesian coordinate system,
thereby, besides inducing a concomitant blood circulation from said supply blood vessel through said blood vessel under treatment, the apparatus ablates said undesirable deposits from a portion of said blood vessel under treatment in proximity to said blood delivering and injecting unit.

2. The apparatus of claim 1 wherein said blood delivering and injecting unit further comprises a series connection of a delivery tube in communicative connection with said blood extracting and pressurizing unit, a secondary manifold and an injector nozzle that, upon its placement into a desired portion of said blood vessel under treatment, effects a forceful ejection of the pressurized source blood into said blood vessel under treatment for ablating said deposits.

3. The apparatus of claim 2 wherein said blood extracting and pressurizing unit further comprises a primary manifold having a primary inlet, a primary outlet and a pumping means connected in between for receiving said source blood through said primary inlet and pressurizing said source blood for delivery to said delivery tube through said primary outlet.

4. The apparatus of claim 3 wherein said blood extracting and pressurizing unit further comprises:
a tertiary manifold having a tertiary outlet and at least one suction needle for piercing said supply blood vessel and drawing said source blood there from; and
a suction tube, in communicative connection with said tertiary outlet and said primary inlet, for suctionally delivering said source blood from said tertiary outlet to said primary manifold.

5. The apparatus of claim 3 wherein said blood delivering and injecting unit further comprises a bendable guide wire axially threaded through said delivery tube, said secondary manifold and said injector nozzle for piercing said blood vessel under treatment and guiding said injector nozzle, said secondary manifold and said delivery tube along said blood vessel under treatment for ablating a corresponding portion of said blood vessel under treatment.

6. The apparatus of claim 5 wherein said secondary manifold further comprises a series connection of an upstream section of delivery catheter, at least one secondary storage chamber, in communicative connection with said delivery tube through said delivery catheter for buffering the pressurized source blood, and an injection catheter for injecting the buffered pressurized source blood into said blood vessel under treatment through said injector nozzle.

7. The apparatus of claim 3 wherein the primary manifold further comprises a primary storage means for temporarily storing said extracted source blood from said supply blood vessel.

8. The apparatus of claim 7 wherein the primary storage means further comprises an upstream pre-pressurized aft chamber and a downstream post-pressurized fore chamber, with said pumping means communicatively connected in between, for storing lower pressure blood within said aft chamber and storing higher pressure blood within said fore chamber.

9. The apparatus of claim 8 wherein the aft chamber further comprises an inline filter for ridding the extracted source blood of undesirable substances.

10. The apparatus of claim 9 wherein said aft chamber further comprises at least one optional power transducer, located upstream of said inline filter, for converting a high frequency power electrical signal of one or more frequencies into a corresponding ultrasonic power emission into the blood to pulverize and emulsify the undesirable substances of the extracted source blood thereby enhancing the effectiveness of said inline filter.

11. The apparatus of claim 10 wherein the geometry of said aft chamber is further tailored to produce a strong resonant standing wave of the ultrasonic power emission so as to maximize the intensity of pulverization and emulsification of the undesirable substances.

12. The apparatus of claim 10 wherein the temperature of said aft chamber is further controlled to be within a pre-determined range conducive to the generation of intense cavitations so as to maximize the intensity of pulverization and emulsification of the undesirable substances.

13. The apparatus of claim 12 wherein said pre-determined temperature range is from about 60° C, to about 80° C.

14. The apparatus of claim 6 wherein said secondary manifold further comprises a power transducer, affixed in proximity to the tip of said injector nozzle, for converting a high frequency power electrical signal of one or more frequencies into a corresponding ultrasonic power emission into the blood to remove the undesirable deposits inside said blood vessel under treatment via pulverization and emulsification during an ablating process to remove said undesirable deposits.

15. The apparatus of claim 14 wherein the supply blood vessel is a downstream section of the blood vessel under treatment whereby the ablated undesirable deposits get immediately collected by said inline filter thus removed from blood circulation.

16. The apparatus of claim 15 wherein said tertiary manifold further comprises at least one optional power transducer, affixed in proximity to the tip of said suction needle, for converting a high frequency power electrical signal of one or more frequencies into a corresponding ultrasonic power emission into the blood to remove the undesirable deposits inside said blood vessel under treatment via multi-stage pulverization and emulsification.

17. The apparatus of claim 14 wherein said primary manifold further comprises an electrical subsystem for:
generating a required electrical drive power for said pumping means; and
generating said high frequency power electrical signal of one or more frequencies for said power transducer.

18. The apparatus of claim 6 wherein said secondary manifold further comprises an electrical discharge means, affixed in proximity to the tip of said injector nozzle, for providing charges to neutralize excess opposite-sign charges generated from the tearing of healthy or diseased tissues during the ablating process.

19. The apparatus of claim 6 wherein said secondary manifold further comprises an electrical discharge means, integrated as part of said injector nozzle, for providing charges to neutralize excess opposite-sign charges generated from the tearing of healthy or diseased tissues during the ablating process.

20. The apparatus of claim 18 wherein said electrical subsystem further comprises an electrical discharge supply circuit for supplying electrical signals and power required by said electrical discharge means.

21. The apparatus of claim 20 wherein said blood delivering and injecting unit further comprises a plurality of conductors, threading through said primary outlet, said delivery tube and said secondary manifold, for interconnecting all electrical systems located at the secondary manifold to their counterparts in the electrical subsystem.

22. The apparatus of claim 21 wherein said plurality of conductors further comprises a waveguide structure for insulating and isolating the interconnecting electrical signal and power lines from one another and from the pressurized source blood.

23. The apparatus of claim 6 wherein said secondary manifold further comprises a drug discharging means, affixed in proximity to the tip of said injector nozzle, for discharging drugs into the blood stream of said blood vessel under treatment.

24. The apparatus of claim 23 wherein the discharged drugs are anticoagulant drugs for preventing a clot formation during the ablating process.

25. The apparatus of claim 23 wherein the ultrasonic power emission, the forceful injection of pressurized source blood and the discharging of drugs into the blood vessel under treatment are sequentially carried out in time to effect a mixed mode ablating process.

26. The apparatus of claim 23 wherein the ultrasonic power emission, the forceful injection of pressurized source blood and the discharging of drugs into the blood vessel under treatment are simultaneously carried out in time to effect a continuous mode ablating process.

27. The apparatus of claim 23 wherein said primary manifold further comprises a drug metering means, communicatively connected to said primary storage means, for supplying and metering auxiliary drugs at a pre-determined rate as desired by the ablating process.

28. The apparatus of claim 27 wherein said auxiliary drugs are the anticoagulant drugs for clot prevention.

29. The apparatus of claim 6 wherein said secondary manifold further comprises a heating means, affixed in proximity to the tip of said injector nozzle, for providing localized heating to destroy diseased tissue during the ablating process.

30. The apparatus of claim 6 wherein said secondary manifold further comprises a heating means, integrated as part of said injector nozzle, for providing localized heating to destroy diseased tissue during the ablating process.

31. The apparatus of claim 6 wherein said secondary manifold further comprises a radio-contrast substance injecting means, affixed in proximity to the tip of said injector nozzle, for injecting radio-contrast substances into the blood stream to enable the examination of said blood vessel under treatment using X-rays.

32. The apparatus of claim 6 wherein said secondary manifold further comprises an ultrasound imaging means, affixed in proximity to the tip of said injector nozzle, for illuminating and examining an illuminated ultrasound image of the blood vessel under treatment.

33. The apparatus of claim 32 wherein, for ultrasonically illuminating the blood vessel interior, said ultrasound imaging means further comprises an imaging frequency ultrasonic transmitter having an input imaging frequency signal as its reference.

34. The apparatus of claim 33 wherein said electrical subsystem further comprises an imaging frequency signal generator for supplying said imaging frequency signal required by said imaging frequency ultrasonic transmitter.

35. The apparatus of claim 6 wherein said at least one secondary storage chamber further comprises a foldable balloon that, upon its inflation under a hydraulic pumping action from said pumping means, substantially blocks the lumen of said blood vessel under treatment within a safety stretch limit while the inflated foldable balloon gets simultaneously pushed along in the Z-direction under the same pumping action.

36. The apparatus of claim 35 wherein said electrical discharge means is affixed to the outside surface of said balloon for providing discharges in close proximity to the diseased or torn healthy tissues to neutralize excess charges generated there from with better efficiency.

37. The apparatus of claim 35 wherein the pumping action from said pumping means simultaneously sends the pressurized source blood through said injector nozzle to create a localized elevated blood pressure while inflating said foldable balloon to prevent an undesirable back flow of the pressurized source blood.

38. The apparatus of claim 35 wherein said foldable balloon, upon cessation of the pumping action from a deactivated pumping means, deflates to allow easy movement of said secondary manifold along the Z-axis.

39. The apparatus of claim 37 wherein the intensity of the hydraulic pumping action is further made adjustable.

40. The apparatus of claim 14 wherein the frequency of a first frequency component of the high frequency power electrical signal is made to periodically vary through a pre-determined range so as to tune the ultrasonic power emission to the various mechanical resonances of the calcified tissue of the undesirable deposits thus further enhancing the ability to shatter and pulverize the calcified tissue.

41. The apparatus of claim 40 wherein said high frequency power electrical signal further includes at least one second frequency component of about equal power while having a frequency that is different from said first frequency component.

42. The apparatus of claim 41 wherein said at least one second frequency component is selected to differ, in frequency, from said first frequency component to generate an ultrasonic power emission having a spatially slowly varying standing wave pattern thereby achieving a more spatially uniform pulverization of the deposited plagues.

43. The apparatus of claim 42 wherein said at least one second frequency component is selected to differ, in frequency, from said first frequency component by less than 10%.

44. The apparatus of claim 42 wherein the power range of each of the ultrasonic power emission and the heating means is at least 1 watt.

45. The apparatus of claim 33 wherein said imaging frequency is made higher than the mean frequency of said ultrasonic power emission to avoid an interference between said ultrasound imaging means and said ultrasonic power emission.

46. The apparatus of claim 45 wherein said imaging frequency is made at least ten times higher than the mean frequency of said ultrasonic power emission.

47. The apparatus of claim 33 wherein said imaging frequency is selected such that the imaging wavelength corresponding to said low power imaging frequency ultrasonic transmitter is less than the mean wavelength of said ultrasound power transmitter to avoid a mutual interference there between and to provide a sufficient image spatial resolution to facilitate the imaging of the wave pattern of said ultrasound power transmission.

48. The apparatus of claim 47 wherein said imaging frequency is selected such that the imaging wavelength corresponding to said low power imaging frequency ultrasonic transmitter is at least ten times less than the mean wavelength of said ultrasound power transmitter.

49. The apparatus of claim 14 wherein the wavelength and power of said ultrasonic power emission are adjusted to generate, within the blood of said blood vessel under treatment, cavitations that preferentially shatter hardened diseased regions based upon their inelasticity while leaving healthy, elastic blood vessel tissues unaffected.

50. The apparatus of claim 49 wherein the wavelength and power of said ultrasonic power emission are further modulated to match a range of natural resonant frequencies of the hardened diseased regions thereby realizing a more effective ablating process.

51. The apparatus of claim 50 wherein said range of natural resonant frequencies is further limited to those of the inelastic diseased region thereby making the ablating process self-terminating in that, once the inelastic diseased regions are removed and flushed away, the corresponding ultrasound pulverization and emulsification actions automatically terminate.

52. The apparatus of claim 51 wherein the radiation pressure exerted by said ultrasonic power emission further propels hardened thus inelastic tissue debris and excises them away from the diseased area.

53. A method for ablating undesirable deposits along the inner blood vessel wall of human and animals, the method comprising:
extracting source blood from a supply blood vessel and pressurizing the extracted source blood; and
delivering and forcefully injecting, through a point of injection, the pressurized source blood into a blood vessel under treatment, wherein the direction of blood flow is designated as Z-direction of a Cartesian coordinate system
thereby, besides inducing a concomitant blood circulation from said supply blood vessel through said blood vessel under treatment, the method ablates said undesirable deposits from a portion of said blood vessel under treatment in proximity to said point of injection.

54. The method of claim 53 wherein pressurizing the extracted source blood further comprises pumping the extracted source blood.

55. The method of claim 53 wherein extracting source blood further comprises piercing said supply blood vessel and drawing said source blood there from.

56. The method of claim 53 wherein delivering and injecting the pressurized source blood into the blood vessel under treatment further comprises piercing said blood vessel under treatment and guiding the pressurized source blood along said blood vessel under treatment to said point of injection.

57. The method of claim 56 wherein delivering the pressurized source blood further comprises buffering said pressurized source blood before injecting said pressurized source blood.

58. The method of claim 54 wherein pumping the extracted source blood further comprises buffering said extracted source blood before pumping it.

59. The method of claim 54 wherein pumping the extracted source blood further comprises buffering said extracted source blood after pumping it.

60. The method of claim 57 wherein buffering the pressurized source blood further comprises introducing an ultrasonic power emission of one or more frequencies, near said point of injection, to remove diseased tissues inside said blood vessel under treatment via pulverization and emulsification during the ablating process to remove the undesirable deposits.

61. The method of claim 57 wherein buffering the pressurized source blood further comprises introducing a discharge of charges, near said point of injection, to neutralize excess opposite-sign charges generated from the tearing of healthy or diseased tissues during the ablating process to remove the undesirable deposits.

62. The method of claim 57 wherein buffering the pressurized source blood further comprises discharging drugs, near said point of injection, into the blood stream of said blood vessel under treatment to prevent a clot formation during the ablating process.

63. The method of claim 58 wherein buffering the extracted source blood before pumping it further comprises, as desired by the ablating process, metering auxiliary drugs at a pre-determined rate into said extracted source blood.

64. The method of claim 63 wherein the auxiliary drugs are anticoagulant drugs for clot prevention.

65. The method of claim 57 wherein buffering the pressurized source blood further comprises providing localized heating, near said point of injection, to destroy diseased tissue during the ablating process.

66. The method of claim 57 wherein buffering the pressurized source blood further comprises injecting a radio-contrast substance, near said point of injection, into the blood stream of said blood vessel under treatment to enable the examination of said blood vessel under treatment using X-rays.

67. The method of claim 57 wherein buffering the pressurized source blood further comprises ultrasonically illuminating and imaging, near said point of injection, the blood vessel under treatment.

68. The method of claim 67 wherein ultrasonically illuminating the blood vessel interior further comprises providing an imaging frequency ultrasonic transmitter having an input imaging frequency signal as its reference.

69. The method of claim 57 wherein buffering the pressurized source blood further comprises providing a foldable balloon that, upon its inflation from pumping the source blood, substantially blocks the lumen of said blood vessel under treatment within a safety stretch limit while the inflated foldable balloon gets simultaneously pushed along in the Z-direction from the same pumping action of the source blood.

70. The method of claim 69 wherein pumping the pressurized source blood through said point of injection simultaneously creates a localized elevated blood pressure while inflating said foldable balloon to prevent an undesirable back flow of said pressurized source blood.

71. The method of claim 69 wherein said foldable balloon, upon cessation of the pumping action of the source blood, deflates to allow easy movement of said point of injection along the Z-axis.

72. The method of claim 60 wherein said ultrasonic power emission further includes a first frequency component and at least one second frequency component of about equal power while having a frequency that is different from said first frequency component.

73. The method of claim 72 further comprises selecting said second frequency component to differ, in frequency, from said first frequency component to generate an ultrasonic power emission having a spatially slowly varying standing wave pattern thereby achieving a more spatially uniform pulverization of the deposited plagues.

74. The method of claim 73 further comprises selecting said second frequency component to differ, in frequency, from said first frequency component by less than 10%.

75. The method of claim 68 further comprises making the imaging frequency to be higher than the mean frequency of said ultrasonic power emission to avoid an interference between said ultrasonic power emission and ultrasonically illuminating and imaging the blood vessel interior.

76. The method of claim 75 further comprises making the imaging frequency to be at least ten times higher than the mean frequency of said ultrasonic power emission.

77. The method of claim 68 further comprises selecting the imaging frequency such that the imaging wavelength corresponding to said low power imaging frequency ultrasonic transmitter is less than the mean wavelength of said ultrasonic power emission to avoid a mutual interference there between and to provide a sufficient image spatial resolution to facilitate the imaging of the wave pattern of said ultrasound power transmission.

78. The method of claim 77 further comprises selecting the imaging frequency such that the imaging wavelength corresponding to said low power imaging frequency ultrasonic transmitter is at least ten times less than the mean wavelength of said ultrasonic power emission.

79. The method of claim 60 further comprises adjusting the wavelength and power of said ultrasonic power emission to generate, within the blood of said blood vessel under treatment, cavitations that preferentially shatter hardened diseased regions based upon their inelasticity while leaving healthy, elastic blood vessel tissues unaffected.

80. The method of claim 79 further comprises modulating the wavelength and power of said ultrasonic power emission to match a range of natural resonant frequencies of the hardened diseased regions thereby realizing a more effective ablating process.

81. The method of claim 80 further comprises limiting said range of natural resonant frequencies to those of the inelastic diseased region thereby making the ablating process self-terminating in that, once the inelastic diseased regions are removed and flushed away, the corresponding ultrasound pulverization and emulsification actions automatically terminate.

82. The method of claim 81 further comprises propelling, with the radiation pressure exerted by said ultrasonic power emission, hardened thus inelastic tissue debris and excising them away from the diseased area.
